(19) United States
(12) Patent Application Publication
(10) Pub. No.: US 2007/0269845 A1
(43) Pub. Date: Nov. 22, 2007

(54) Method of Generating and Isolating Tumour Cells

(75) Inventor: Rolf Bjerkvig, Mathopen (NO)

Correspondence Address: SEED INTELLECTUAL PROPERTY LAW GROUP PLLC, 701 FIFTH AVE, SUITE 5400, SEATTLE, WA, 98104, US

(73) Assignee: Cytovation AS, Bergen (NO)

(21) Appl. No.: 10/555,297

(22) PCT Filed: Apr. 29, 2004
(86) PCT No.: PCT/GB04/01866

§ 371 Date: Jul. 11, 2007

(30) Foreign Application Priority Data
May 2, 2003 (GB) 0310191.2

(51) Int. Cl. C12N 5/06; C12Q 1/02

(52) U.S. Cl. 435/29; 435/325; 435/366

(57) ABSTRACT

The invention provides a method of generating and isolating cells of a defined tumour phenotype being invasive and angiogenesis-independent (phenotype I), from a tumour sample, said method comprising the steps of culturing tumour cells of said tumour sample for up to nine days in order to establish multicellular spheroids, and implanting said multicellular spheroids thus obtained into an immunodeficient animal. It further provides methods for generating and isolating tumour cells of phenotype II (invasive and angiogenesis independent) which involves the generation of cells of phenotype I and serial passaging of the cells in animals. Furthermore, by modifying the amount of time the spheroids are cultured prior to implantation into an animal, cells of phenotype III (non-invasive, angiogenesis dependent) may be generated and isolated. The present invention further provides animal models of all phenotype tumours, isolated tumour cells of the defined phenotypes and uses thereof.

The present invention relates to methods for generating and isolating cells of one or more defined phenotypes from a malignant tumour. Particularly, the method of the invention can be used to isolate transformed stem cells from a malignant tumour, particularly a brain tumour.

Cancer is a class of disease caused, in many cases, by the growth of a malignant tumour within the body of a patient. Abnormal and uncontrolled cell division occurs to form the malignant tumour, which may invade and destroy the tissues in which it arises. Malignant tumours, or cancer, can arise in almost any tissue, including but not restricted to lung, bronchi, stomach, breast, colon, prostate gland, brain, liver, pancreas, kidney and skin. Cancer can thus arise from any cell type in the body, and is one of the major causes of human morbidity. Cancer can be defined as an inappropriate, excessive, and continuous proliferation of transformed cells. Malignant tumours are thought to arise from one ancestral cell, and can thus be described as “monoclonal”, and all cells of the tumour are descendants of the ancestral cell. The ancestral cell undergoes a transformation into a cancer-cell, proliferates and produces the population of cells recognised as a tumour. As the malignant tumour develops, the cells of which it is composed may acquire new traits and thus become different from one another. Thus, the malignant tumour may contain distinct subpopulations of cells.

Stem cells, “generic” or pluripotent or multipotent cells that can make copies of themselves indefinitely, are known to be present in various organs within the human or animal body. These cells have the potential to produce specialized, differentiated cells, and can thus replace dying cells and repopulate injured or diseased areas within an organ. Thus, stem cells are undifferentiated cells which retain the ability to differentiate into a particular specialized cell e.g. bone marrow stem cells into blood cells.

It has recently been suggested that pluripotent stem cells represent the initial and key cell population within a tissue or organ for the development of malignant tumours. Thus, the ancestral cell from which the tumour develops may arise or originate from the stem cell population, which stem cells have the ability to perpetuate themselves via self-renewal. It is also hypothesized that tumours may contain “cancer stem cells”, rare cells with an indefinite potential to proliferate. Such cells are discussed in Reya et al., Nature, Vol 414, November 2001, pages 105 to 111, and may be descendants of a transformed stem cell.

Thus, transformed stem cells may represent a self-renewing cell population that may be found in certain malignant tumours, or in malignant tumours, as they originate. This cell population may be a key cell population from which heterogeneic tumour cells may develop. Thus, as a tumour develops, many different, i.e. heterogenous (or heterogeneic) tumour cells may arise to make up the tumour (e.g. cells within the tumour may differentiate). In established tumours, it has been thought that the bulk of the tumour is made of heterogeneic tumour cells and thus the tumour has a largely heterogeneic phenotype. It is this heterogeneic phenotype that provides the bulk of the information upon which the tumour is histopathologically identified.

Recently it has been shown that neural stem cells transplanted into the adult brain show extensive infiltration within the central nervous system (CNS), a trait that is also shared by malignant brain tumours. This raises the question of whether stem cells can give rise to brain tumours. It has been shown that brain tumour cells can express a variety of antigens shared by developing neural stem cells, e.g. the intermediate filament proteins nestin and vimentin (Dahlstrand et al., Cancer Research, 1992, 52(9), pages 5334-5341 and Salinen et al., Cancer Research, 2000, 60(23), pages 6617-6622), the NG2 proteoglycan and specific gangliosides. Biochemical analyses of autopsy brains from individuals diagnosed with brain tumours have shown that brain areas invaded by tumour cells contain relatively large amounts of the gangliosides 3′-isoLM1 and 3′6′-iso1LD1 (Wilkstrand et al., Prog Brain RS, 1994, 101, pages 213-223). These gangliosides are not expressed in the normal adult brain (after two years of age), but are found during brain development and are closely linked with glial proliferation and migration, with the highest peak (10 nmol sialic acid/g tissue) during the first trimester (von Holst et al., Acta Neurochir, 1997, 139, pages 141-145; Fredman et al., J Neurochem, 1993, 60(1), pages 99-105; Sung et al., Cancer, 1994, 74(11), pages 3010-3022). This may imply that 3′-isoLM1 has a function during neural as well as tumour cell migration.

Another marker expressed by brain tumour cells is the NG2 proteoglycan. NG2 is known to be expressed during embryogenesis as early as embryonic day 12, and is especially associated with brain capillaries (Oohira et al., Arch Biochem Biophys, 2000, 374(1), pages 24-34). NG2 is expressed throughout the period of rapid expansion of the brain vasculature and is down-regulated as the vessels terminally differentiate (Diers-Fenger et al., Glia, 2001, 34(3), pages 213-228). In the adult CNS, oligodendroglial precursor cells also express NG2 (Shoshan et al., Proc. Natl. Acad Sci USA, 1999, 96(18), pages 10361-10366). The present inventors have recently shown that overexpression of NG2 increases tumour initiation and growth rates, neovascularization and cellular proliferation, which predisposes to a poorer survival outcome (Chekenya et al., Faseb J, 2002, 16(6), pages 586-588). Quite recently it has also been shown that human glial tumours may have neural stem-like cells expressing astrological and neuronal markers in vitro (Ignatora et al., Glia, 2002, 39, pages 193-206).

Based on these observations, the present inventors postulate that it is likely that neural precursor cells actually represent the normal counterpart of brain tumour cells capable of migration. The migratory behaviour of brain tumour cells can be explained by a predisposed interplay between normal brain tissue and the migrating cells where the brain represents a permissive tissue guiding cells with certain phenotypic traits to migrate along specific anatomical structures. In this context, it should be emphasised that the presence of multipotent cells in specific brain regions (like the subventricular zone) correlates well with the distribution and differentiation capacity of a plethora of brain tumours.

The present inventors thus believe and propose that tumour cells expressing stem cell characteristics are important in tumour development, and thus are important to study with a view to understanding tumour development and/or developing effective therapies against tumours. This underlies the present invention. In particular, it is believed that the initial transformation event that leads to the development of cancer, e.g. generation of a tumour, arises in a stem cell. This transformed stem cell thus represents the “origin” cell of a cancer or tumour.

In order to understand the mechanisms that occur in tumour generation, perpetuation and growth, the study of tumour cells in vitro and animal models is of great clinical value. Such studies permit a greater understanding of the processes that occur in tumours, the changes the tumour cells undergo, an understanding of the genetic changes and alterations in protein expression patterns and ultimately provide a research tool or experimental model to investigate new therapies and methods of tumour ablation.

Tumours are normally a heterogeneous population of tumour cells, since the cells may differentiate and/or acquire new mutations as they rapidly divide and propagate. Thus, when tumours are isolated from their in vivo position, they can contain numerous “subpopulations” of tumour cells, each with different properties or genetic expression profiles. It is of particular interest in the field to be able to isolate or generate from tumour samples homogeneous cell populations which can be compared against one another in order to get a clearer picture of the changes that occur during tumour progression and to identify particular drug targets, for example to devise particular ablation compounds and techniques that can target one or more particular cell populations.

Of particular interest in this area would be the “transformed stem cells” as discussed earlier. Such cells are thought to have indefinite proliferative potential that may drive the formation and growth of tumours. Thus, some of the other cell types present within a tumour that are more differentiated may lose the ability to proliferate extensively. It is therefore of clinical interest to be able to isolate and study the “transformed stem cells” in particular, together with the other tumour cell types.

The present invention thus aims to provide methods for generating and/or isolating particular cell types from a tumour sample, particularly for isolating “transformed stem cells” from tumour tissue.

Angiogenesis (new blood vessel formation) is generally a prerequisite in the growth and development of tumours. A blood supply to the tumour provides a source of nutrients, a means for removal of waste products and an avenue for metastasis. Thus in order to grow larger, the tumour needs to stimulate new blood vessel formation. The tumour cells stimulate a multifunctional cascade of events in order to promote proliferation and differentiation of endothelial cells, which leads to angiogenesis.

A further characteristic of many tumours is their ability to invade the tissue surrounding the tumour site. The result of tumour cell invasion is the destruction of the surrounding healthy tissue. Invasion involves the degradation of basement membranes and complex interactions with the extracellular matrix of adjacent cells. The matrix metalloproteases appear to be essential enzymes for tumour cell invasion, however, the mechanisms by which invasion occur are still poorly understood. Invasion is characterised by a frontier of malignant tumour cells that are progressively destroying the surrounding normal tissue. Invasion is a complex multi-factorial process which is influenced by stimuli in the surrounding cellular environment and is modulated by interactions between different cell types.

The present inventors have identified for the first time three specific phenotypes of tumours or tumour cells that can be isolated or generated from excised tumour tissue. The three tumour or tumour cell types can be defined by their angiogenesis dependence and invasive capacity. One of these phenotypes, Type I cells, have been found to exhibit stem cell characteristics, e.g. to express stem cell markers, and are believed to comprise or represent “transformed stem cells”. Such “transformed stem cells” have the capacity to repopulate the tumour, and may represent the core source of cells for tumour development. Tumours of type I cells are highly invasive, but do not depend upon angiogenesis for growth (i.e. are angiogenesis independent). This is a new characteristic, identified for the first time in the present invention. The existence and/or importance of such a cell population had previously not been recognised or appreciated. The present inventors have shown that this cell population expresses one or more stem cell markers and thus may also be defined as “transformed stem cells”. These “transformed stem cells” are proposed herein to represent the “originating” cell for tumour development.

The observation, reported for the first time herein, that tumours may have the capacity for angiogenesis-independent growth, mediated by a sub-population of transformed stem cells (“cancer stem cells”) which show invasion and cell division between existing vasculature, challenges the generally-accepted and current view of tumour growth as an angiogenesis-dependent process.

Tumours and cells of “phenotype II” have also been identified and isolated by the inventors of the present invention. Such cells express a reduced number of stem cell markers when compared to cells of phenotype I. Further, tumours of these cells are invasive, and are dependent upon angiogenesis for growth (i.e. are angiogenesis-dependant).

The inventors have further isolated a cell population referred to as “phenotype III” which also have a reduced number of stem cell markers when compared to cells of phenotype I. The tumours of these cells are non-invasive but are dependent on angiogenesis for growth.

The inventors of the present invention have not only identified the above-mentioned tumours and cells, but have devised methods for generating clinically relevant animal models comprising such cells or tumours, and additionally methods of isolating cells of the different phenotypes, advantageously from a single tumour biopsy.

In this regard the methods of the invention for generating and isolating cells of a particular phenotype, depend upon establishing in an animal host tumours generated from clinical tumour tissue samples (e.g. tumour biopsies). It has been found that the manner in which this tumour in the host animal is generated may influence or dictate the nature of the tumour obtained i.e. whether it is of phenotype I, II or III. This step thus leads to the generation of an animal model for the tumour type concerned (depending on which conditions and/or methodologies are adopted). The cells of the desired phenotype may then be isolated from the tumour, more generally from the tissue or organ of the animal model (i.e. the animal model may provide the source for isolation of the tumour cells that have been generated within it).

In particular, it has been found that tumours or cells of the phenotypes mentioned above can be obtained from an excised tumour if cells from the tumour are cultured in vitro for a specified amount of time, and/or under particular conditions, and then implanted into an animal. The implanted cells develop into a tumour in the host animal, from which cells of a particular phenotype may be isolated. Primary or first generation tumours developed in this way may also be used to study tumour progression, as described further below.

The methods of the invention rely on culturing tumour cells in vitro prior to implantation, in order to obtain structures known as spheroids. The spheroids are then implanted. In particular, it has been found that the length of culture of the cells prior to implantation is important in determining the type of tumour which is obtained. This is a new observation, not previously reported, and underlies the ability of the methods of the invention to be used, reliably and precisely, to obtain tumours of a particular phenotype of choice. Advantageously, this then permits tumours of different phenotype readily to be compared.

Spheroids are three-dimensional multicellular structures, well known in the art to be formed by cancer or tumour cells (and other cells) in culture. Spheroids may be formed from monolayer cells in culture, when these are grown by various in vitro culture methods, as known in the art and described in the literature, and have been widely used as model system for studying three-dimensional growth and differentiation in vitro, or in investigating cell-cell interactions, drug effects etc. in vitro.

The new methods of the invention thus provide animal models with a tumour, derived from implanted spheroids, of a known tumour cell phenotype and methods of generating and/or isolating substantially homogeneous cells of a known phenotype. It has not previously been taught or suggested that implantation of tumour cells into laboratory animals can result in the generation of an animal model containing substantially only one phenotype of tumour cell (i.e. a substantially homogenous tumour), nor the isolation of substantially one phenotype of cell, from a heterogeneous tumour sample. These animal models and cells can thus be analysed, and allow for better understanding of the tumour involved and the development of effective treatments for the tumour.

The method is also particularly suited to the use of the animal models or the tumours or isolated cells in comparative studies, such as comparison of differentially expressed proteins (for example, proteins differentially expressed as between the three different phenotypes), and use of the animal models or tumours or cells as tools in drug discovery.

Accordingly, in one aspect, the present invention provides a method of generating cells of a defined tumour phenotype, being invasive and angiogenesis-independent (phenotype I), from a tumour sample, said method comprising the steps of culturing tumour cells of said tumour sample for up to nine days in order to establish multicellular spheroids, and implanting said multicellular spheroids thus obtained into an immunodeficient animal.

The above-mentioned method results in an animal with implanted tumour cells. The cells grow and develop into a tumour, resulting in an animal containing an experimentally-derived tumour i.e. an animal model. The above-mentioned method thus involves allowing said implanted spheroids to develop into a tumour. This tumour will contain cells of the defined phenotype, phenotype I. The method may thus also be viewed as a method of generating a tumour of phenotype I. The present invention extends to the animal model thus derived.

Therefore, in a related aspect, the present invention provides a method of generating an animal model of a defined tumour phenotype being invasive and angiogenesis-independent (phenotype I tumour) from a tumour sample, said method comprising the steps of culturing tumour cells of said tumour sample for up to nine days in order to establish multicellular spheroids, and implanting said multicellular spheroids thus obtained into an immunodeficient animal.

The method thus involves or includes allowing the implanted spheroids to develop into a tumour.

The invention further extends to an animal model obtainable by the method of the invention.

Tumour cells of phenotype I may be isolated from the animal by standard methods, as discussed further below, for example by removing or excising the tumour from the animal and isolating the cells therefrom, or by directly isolating the cells from the animal or the animal tissue or organ in which the tumour has developed.

Thus, in a further aspect, the present invention also provides a method of isolating cells of a defined tumour phenotype being invasive and angiogenesis-independent (phenotype I) from a tumour sample, said method comprising the steps of culturing tumour cells of said tumour sample for up to nine days in order to establish multicellular spheroids, implanting said multicellular spheroids thus obtained into an laboratory animal, and isolating tumour cells of said phenotype from said animal.

Cells of phenotype I isolated, or obtainable, by the above-described methods form a further aspect of the present invention.

More particularly, in this aspect the method of generating cells of tumour phenotype I can comprise the following further steps:

allowing a tumour to develop in said animal from said implanted spheroids (e.g. monitoring said animal until symptoms of tumour presence occur, or simply maintaining (i.e. holding or keeping) said animal for a time period suitable to allow said tumour to develop) optionally sacrificing said animal, and isolating tumour cells therefrom. For example, the tumour or tissue of said tumour may be excised (or removed or isolated) from said animal, and the cells isolated therefrom.

The cells may be isolated as discussed further below.

The term “phenotype I” as used herein defines tumours or tumour cells that are invasive and angiogenesis-independent. By “invasive” is meant that the tumour, or the tumour from which the cells derive, is able to divide, invade, or infiltrate, surrounding cells or tissue. In particular, tumours of phenotype I have been shown to be highly invasive. Thus, they do not grow as discrete or localised lesions, but are diffusive, i.e. infiltrated into surrounding tissue. The tumours may exhibit an ill-defined or no defined host/tumour border. Thus, the tumour may be poorly circumscribed. A disseminated spread of tumour cells may be seen in the host tissue.

By “angiogenesis-independent” is meant that the tumour, or the tumour from which the cells derive, does not require angiogenesis (i.e. the development of new blood vessels) to grow and/or survive. Thus, cells of an angiogenesis-independent tumour may grow and divide between normal blood vessels present in the tissue, i.e. between existing vasculature. Such angiogenesis-independent tumours may co-opt the host vasculature. In this way such a tumour may present as an aggressive disease without angiogenesis (i.e. without the growth of new blood vessels).

As described further below, the characteristics of invasiveness and angiogenesis-dependence can readily be determined by known or standard methods, for example by studying the morphology of the resulting tumour (e.g. by visual (e.g. macroscopic) or microscopic inspection), by histological techniques or methods (e.g. immunohistochemistry or other staining techniques)), e.g. in samples or sections of the resulting tumour or indeed in the intact tumour itself, for example in situ in the animal by imaging or scanning methods e.g. MRI. Angiogenesis-dependence can be observed morphologically or histologically, e.g. by looking for tumour vasculature and formation of new blood vessels, and/or necrotic regions. The vasculature (e.g. the morphology) of the tumour can be compared to that of corresponding normal tissue (e.g. in a control animal or in unaffected or non-tumoural areas of the host animal), for example microvessel density (MVD) or vascular area. Functional comparisons may also be made, e.g. perfusion and hypoxia studies, or by studying the expression and/or distribution of endothelial cell markers (e.g. CD31 and von Willebrand factor) or VEGF, or other vascular growth factors.

As mentioned above, the tumour cells of phenotype I are further believed to be transformed stem cells. In particular, they have been shown to express one or more stem cell markers. The tumour cells of phenotype I have further been shown to have a self-renewal capacity. Thus, the “transformed stem cell” phenotype can be defined upon the basis that the cell expresses at least one stem cell marker, and is capable of self-replication (or self-renewal). The presence of such cells can be ascertained by transferring the resulting tumour tissue or cells extracted from the animal into a serum-free stem cell medium containing epidermal and fibroblast growth factors. Cells that grow in such medium are transformed stem cells. The cells can also be tested for expression of stem cell markers which are dependent on the tissue type from which the tumour is derived, for example nestin is primarily a brain tissue stem cell marker. Thus, cells of phenotype I isolated (or obtained or generated) from a brain tumour may express one or more neural stem cell markers (e.g. neuronal and/or astroglial stem cell markers).

It has further been shown that tumour cells of phenotype I may exhibit a migratory behaviour similar to normal stem cells. Thus, cells of phenotype I are capable of migration. In particular, the cells can migrate without angiogenesis. The migratory pattern or behaviour of the cells may be studied or investigated as described further below.

It will be understood from this therefore, that by isolating cells of phenotype I, one may isolate transformed stem cells from a tumour.

In a further, related aspect, the present invention thus also provides a method of generating a transformed stem cell from a tumour sample, said method comprising the steps of culturing tumour cells of said tumour sample for up to nine days in order to establish multicellular spheroids, and implanting said multicellular spheroids thus obtained into an laboratory animal.

As above, in this method the implanted spheroids are allowed to develop into a tumour which contains the transformed stem cells.

To isolate transformed stem cells from a tumour sample, such a method may further include the step of isolating transformed stem cells from said animal. Again such transformed stem cells isolated, or obtainable, by the above-described methods form a further aspect of the invention.

This may readily be achieved by standard and well known means, for example by removing or excising the resulting tumour or tissue from the animal and culturing it in a stem-cell specific culture medium, i.e. a culture medium designed to support the growth only of stem cells (e.g. serum-free stem cell medium containing epidermal and fibroblast growth factors). Techniques based upon the use of stem cell specific markers may also be used e.g. immunological or antibody-based separation techniques e.g. immunoaffinity binding, or immunomagnetic separation or FACs sorting etc. Since the cells of phenotype I which are obtained are substantially homogenous (i.e. the phenotype I tumour will be composed substantially of transformed stem cells only), the transformed stem cells may also be isolated by any technique designed or adopted to isolate tumour tissue from the animal, i.e. which can distinguish the tumour tissue in the animal from the normal tissue, as described further below.

A “tumour sample” according to the present invention can be any sample of any tumour. Generally, however, it will be a clinical sample e.g. a biopsy sample, for example collected when the tumour is excised from a patient. Said sample can thus be the entire tumour excised from a patient, or a portion, fragment or part thereof. As described further below the tumour may be of any tumour type and may be obtained from any desired patient e.g. an animal (e.g. mammal) or a human patient. Preferably, the tumour arises as a product or symptom of disease (i.e. cancer) (e.g. spontaneously) rather than being artificially or experimentally induced e.g. in an animal model or in in vitro or culture system (e.g. tumour cells in culture or a tumour cell-line etc.) but the latter are not precluded.

It will be appreciated by the person skilled in the art that a suitable sample or specimen can be taken from a biopsied tumour, and the sample should be selected in such a way to avoid necrotic (dead) tissue, and to select a sample or specimen of a suitable size and nature for the method. The term “biopsy” will be understood to mean the removal of a sample of living tumour tissue from the patient. It is generally understood that the sample is taken from a living patient, but post-mortem extraction is also envisaged if the tissue is extracted as soon as possible after death.

The patient from which the tumour sample is obtained is generally a human patient, since the investigation of human tumours is of most pressing interest in the field. However, as mentioned above the method of the application may be used for any tumour sample, whatever origin, e.g. any animal. Preferably, the tumour sample is freshly obtained (e.g. excised) from a human patient. However, it will also be appreciated that the tumour sample may be treated in any convenient or desired way prior to the culturing step of the present invention, e.g. in chilled or frozen storage etc. in any appropriate medium etc. The tumour sample may be cut into pieces, e.g. 1 mm pieces and stored in serum supplemented growth medium containing the appropriate cryoprotectants, such as dimethylsulfoxide or glycerol. It will of course be understood that the excision step is not necessarily within the scope of the present invention, and that the method of the invention may therefore be performed using ex vivo cells.

As used herein the term “tumour” refers to any population (e.g. solid mass) of malignant cells that are growing in an unwanted and uncontrolled way within the patient. As mentioned previously, tumours can arise in almost any tissue, and any such tumour falls within the scope of the present invention including both solid and other tumours e.g. haemopoietic tumours. Thus the tumour may be from any tissue or organ, and may be of any type e.g. epithelial tissue tumours (carcinomas) or any other type as for instance sarcomas. Thus, for example, tumours that are found in the brain, head, neck, thyroid, mouth and throat, lung, bronchi, oesophagus, stomach, colon, rectum, liver, kidneys, spleen, pancreas, prostate gland, breast, ovary, testicles, endometrium, cervix, skin, muscles, bone or any part of the body are within the scope of this invention. Any tumour that can be biopsied or excised may be used. As used herein, “tumour” refers only to malignant, not benign tumours, e.g. the tumours that cause cancer.

In the method of the invention, the tumour sample is preferably transferred to an aseptic culture medium, preferably Dulbecco's Modified Eagle's Medium (DMEM) (BioWittaker, Verviers, Belgium or Sigma, St. Lois. MA) and fragmented, for example by using a scalpel to cut the sample into pieces. Generally, the pieces are up to 5 mm³, preferably up to 3 mm³, preferably up 1 mm³, preferably 0.1 to 0.5 mm³ in size. The fragmented tumour sample is then cultured in order to obtain spheroids, according to any known or desired technique but preferably by culturing in overlay culture medium. In the culture overlay technique, the biopsy fragments are transferred to culture flasks base coated with agar dissolved in minimal essential media (MEM) with additional proteins, if required. The agar is overlayed with a suspension of minimal essential media plus additional components, if required. The agar overlay suspension flasks are kept at standard tissue culture incubator conditions for the time specified for the method of the invention (for phenotype I, up to 9 days). Such conditions are generally at 100% relative humidity, 95% air and 5% carbon dioxide.

The tumour sample is cultured in the agar overlay suspension in order to form spheroids. “Spheroids” are a solid mass of tumour cells, and their formation usually implies an initial aggregation of cells which then grow into larger, three-dimensional structures, composed of multiple tumour cells. Spheroids are thus three-dimensional aggregates of tumour cells, generally expressing histotypic organisation in vitro comparable to tissue continuity in vivo. The person skilled in the art can ascertain various cell culture techniques, including the agar overlay technique, spinner flask and gyratory rotation systems, for preparing suitable spheroid preparations. However, the agar culture overlay technique is preferred.

The spheroids are cultured for up to nine days prior to implantation, in order to obtain cells of phenotype I. I.e. the duration of the culture period used to obtain the spheroids is up to 9 days, for example up to 9 days post-sampling, or up to 9 days after first placing the tumour cell into culture. The tumour cells may thus be cultured for 1 to 9 days prior to implantation, i.e. 1, 2, 3, 4, 5, 6, 7, 8 or 9 days, or any period up to and including 9 days (e.g. 3 to 9 days). Preferably, the cells are cultured for 5 to 9 days, i.e. 5, 6, 7, 8 or 9 days. More preferably, the cells are cultured for up to 7 days (e.g. 3 to 7 days or 5 to 7 days). The cells are cultured as spheroids, as mentioned previously. Culture conditions are selected in order to promote and maintain spheroids, as discussed previously. The spheroids are passaged in culture if necessary. Generally, the culture medium is changed after 7 days, and thus the cells are “passaged” into fresh culture medium. The culture conditions are as defined previously.

As mentioned above, the culture time used to obtain the spheroids is important in determining which tumour phenotype is obtained. Thus, to obtain tumours of phenotype I a relatively short culture time of up to 9 days is selected. As will be described in more detail further below, longer culture times of period of weeks (e.g. of about 6 weeks) result in the development of tumours of phenotype III when the cultured spheroids are implanted.

The time of culture is also important in determining the reliability and/or specificity of the method. Thus, for example, it has been found that as the culture time of 9 days is increased, cells/tumours of phenotype I may be obtained with decreasing specificity (i.e. the resulting tumours may contain cells of other phenotypes, beyond (i.e. in addition to) phenotype I) and that the heterogeneity of the resulting tumour may increase with increasing spheroid culture time. The present inventors have determined that with a culture period of up to 9 days, a tumour that is substantially homogenous with respect to Type I cells may reliably and consistently be obtained. However, longer cultures may nonetheless be possible to obtain tumours or cells of phenotype I e.g. up to 11 days, up to 15 days or up to 21 days (e.g. 1 to 21, 1 to 15, 1 to 11, 1 to 10, 3 to 21, 3 to 15, 3-11 or 3-10 days).

Once the tumour biopsy has been cultured for the specified (or desired) amount of time, and spheroids obtained, these spheroids are then implanted into an immunodeficient animal. By “immunodeficient” it is meant that the animal has a reduced, or non-functioning immune system, has been immunocompromised, or the immunity has been reduced. Such animals include those that have T-cell deficiencies, as well as those that have both B and T-cell deficiencies. The latter immunodeficient animals are termed SCID (Severe combined Immunodeficient animals). The animal may be any non-human animal, e.g. any non-human mammal. However, laboratory animals are generally preferred e.g. rodents, cats, dogs, monkeys, etc. Although any immunodeficient laboratory animal may be used in the method of the invention, it is preferred to use rodents. Such rodents include rats, mice, guinea pigs, hamsters and gerbils. Immunodeficient rats and mice are preferred.

The spheroids are thus transplanted into the immunodeficient animal. Generally, this implantation, or transplantation step will involve xeno-transplantation, since in the preferred embodiments of the invention human tumour samples will be used, and the resulting spheroids will be implanted into a non-human animal. However, this is not necessarily always the case, since in the case of a tumour sample from a non-human animal, the spheroids may be implanted into an animal of the same species. The spheroids can be transplanted into any part of the animal. Transplantation can take place by any suitable means, the preferred method being direct implantation of the spheroids into the animal. Preferably, the spheroids are transplanted into an organ in the animal, i.e. brain, liver, kidneys, stomach or lungs. A highly vascularised organ (such as liver or brain) is preferred. More preferably, the transplant is orthotopic, wherein the spheroids are implanted in the same organ or tissue as the organ or tissue from which the spheroids were derived. Thus, for example, spheroids derived from brain tumours are implanted into an animal brain, spheroids derived from pancreatic tumours are implanted into the pancreas of an animal etc.

For transplantation, it is preferred that the spheroids used are up to 400 μm in diameter, preferably 100 to 300 μm in diameter, more preferably 200 to 300 μm in diameter. The spheroids can be selected using a micropipette and a stereomicroscope with a calibrated reticle in the eyepiece. Any suitable number of spheroids are selected for implantation, preferably up to 20 spheroids are used, more preferably up to 15, even more preferably about 10 spheroids are implanted (5 to 15, 8 to 12 or 9 to 11). The spheroids may be transplanted together with culture medium, i.e. DMEM.

When the spheroids are transplanted via injection, it is preferred that a Hamilton syringe is used.

During transplantation, the animal is anaesthetised. Prior to and after transplantation, the animals are kept in a pathogen-free environment, since they are immunodeficient.

After transplantation, the animals are generally monitored daily for symptoms of tumour growth (specifically first generation tumour growth). Such symptoms will depend upon the nature of the initial tumour sample and/or the site of transplantation in the animal. The tumours may take several weeks or months to develop, e.g. up to 2, 3, 4, 5, 6, 7 or more months. If the spheroids are transplanted into the brain, symptoms include passivity, clumsiness, weight loss, fatigue and/or paresis or hemiparesis. At other sites, e.g. liver, the symptoms may include weight loss, jaundice, loss of implant site activity. Alternatively, the animal may be examined for tumour growth e.g. by visual inspection, palpation, imaging or scanning techniques etc. Thus growth of a tumour establishes an animal model. The animal model may be used directly e.g. to study the tumour, or the effects of various agents or therapies thereon. Alternatively, or additionally, it may be used further to obtain an associated tumour or tumour cells. Thus, once such symptoms have been observed, the animal may be sacrificed and the organ or tissue containing the tumour, or the tumour itself, may be excised. Alternatively, the animal is sacrificed after a period of 1 to 6 months, e.g. 1 to 5, 1 to 4, or 1 to 3 months, 2 to 6, 2 to 5 or 2 to 4 months, after transplantation (or any time period suitable for a tumour to develop) and the organ or tissue containing the tumour or the tumour itself may be excised. After excision, the tissue may be enzymatically or mechanically dissociated and suspended in media.

Cells of phenotype I can be isolated from the animal, e.g. from the excised organ or tissue by any suitable means known in the art. If the tumour spheroids are derived from a human, a preferred method is to use a pan anti-human antibody to isolate human cells from the animal tissue. Any analogous technique using a pan anti-species antibody may be used to isolate the tumour cells from any xeno-transplantation situation. However, any technique using spheroid-specific or tumour-specific antibodies (or other specific binding partners) may be used to separate the tumour cells from the cells of the animal. Particularly preferred methods of separating the tumour cells from the animal cells include flow-cytometric cell sorting techniques (e.g. fluorescence-activated cell sorting FACs) and magnetic bead separation techniques, wherein an anti-tumour source antibody (or other specific binding partner) is immobilised on magnetic beads, allowing capture of the tumour cells. In the flow-cytometric cell-sorting technique, a fluorescent dye for example may be attached via an antibody (or other binding partner) specific for the tumour or animal cell, and the fluorescent-activated cell-sorter can separate the cells based upon whether the cell has a label or not, and the sorted cells can then be maintained in culture.

Once isolated from the animal, the tumour cells may be maintained in culture. Isolated tumours may also be maintained in culture for limited periods of time, as known and described in the art. Any appropriate culture medium may be used. For example, in the case of Phenotype I the cells may be maintained in growth medium designed for neural stem cells if the tumour cells are derived from a brain tumour for example. Such medium may consist of DMEM/F12 medium, 20 ng/ml BFGF, 20 ng/ml EGF (both R&D systems), 1.5 mM L-glutamine (Gibco), N2 supplement (Gibco).

Thus, using the method of the invention, a substantially homogeneous (i.e. 75%, 80%, 85%, 90%, 95%, 97%, or 99% or more, preferably 90% or 95% or more content of cells of phenotype I) population of cells of phenotype I may be obtained, and represents a further aspect of the invention. Such cells may be maintained in culture and used for studies such as determining patterns of gene expression in these cells, determining or assessing cell ablation techniques and comparing the characteristics of these cells against other tumour cell phenotypes such as phenotypes II and III, or against normal (i.e. non-tumour) cells.

As mentioned above, the method of isolating cells of tumour phenotype I according to the invention, may also be used to isolate transformed stem cells (i.e. tumour stem cells), using cell isolation techniques designed to isolate stem cells specifically, or any of the techniques used to isolate phenotype I cells.

As mentioned previously, the invention also extends to the animal model comprising a tumour composed of cells of phenotype I. Such an animal model is obtained as described previously with regard to the isolation of cells of phenotype I, but is not sacrificed. Instead, the animal model may be studied in order to obtain useful information upon the tumour progression and phenotype. The animal is a clinically relevant model of the tumour, and allows experimental studies upon that tumour to take place. Such studies include studies of tumour biology such as invasiveness and response to experimental therapy.

The tumours or isolated cells of phenotype I can further be used to generate tumours or cells of phenotype II. The term “phenotype II” as used herein refers to tumours or tumour cells that are invasive and angiogenesis-dependent. By “angiogenesis-dependent” is meant that the tumour, or the tumour from which the cells derive, requires angiogenesis to grow and/or survive. Such cells express a reduced number of stem cell markers in comparison to cells of phenotype I, and are thus no longer ‘transformed stem cells’. It is thought by the inventors that such cells represent more differentiated tumour cells that can be found within a tumour. The present invention provides a method of generating tumours or tumour cells of phenotype II from a tumour sample. Such additional method steps form a further embodiment of the invention, and allow tumours and/or cells of phenotype I and phenotype II to be generated from the same tumour sample, allowing a direct comparison of gene expression, histology, morphology and other characteristics such as drug susceptibility between cells of different phenotype generated from the same tumour. Such studies are of great importance to develop an understanding of tumours in situ which are a heterogeneous population of tumour cells. The tumour can thus be targeted as a whole in order to successfully treat the disease.

Tumours or cells of phenotype II can be obtained by serial transplantation and culturing steps, starting from a phenotype I tumour. Thus, as a first step a phenotype I tumour is established from a tumour sample. Cells from said tumour sample are cultured to form spheroids, implanted into immunodeficient animals, and allowed to develop into tumours (e.g. a first generation or phenotype I tumour). Subsequently the resulting tumour cells are isolated from the animal, and the process is repeated until tumours (and cells) of phenotype II are obtained. The tumour cells are thus serially passaged in vivo, with an intermediate step of culturing as spheroids between implantation events. During the serial passaging events, the cell type progressively changes between phenotype I and phenotype II, and thus tumours, or cells of an “intermediate” or “mixed” or “transitional” phenotype may also be obtained. The phenotype progressively changes during the serial passaging until cells of phenotype II are obtained. Thus, the tumour cell type may progressively change from type I to type II. Further, the tumour itself may progressively change from a type I to a type II tumour. Thus, the tumour may progressively gain cells of type II and lose cells of type I, resulting in “mixed” or “intermediate” or “transitional” tumours which may also contain cells of both types. Such a mixed or intermediate or transitional tumour may also contain cells of a mixed or intermediate or transitional phenotype.

Thus, serial animal passages may gradually transform tumours (tumour cells) of phenotype I into an angiogenesis-dependent phenotype (phenotype II). It may be seen, therefore, that invasion and angiogenesis may be uncoupled. Thus, tumours derived from the tumours of phenotype I (which may be seen as stem cell tumours) develop angiogenesis-dependency. This may occur progressively or gradually. The onset of angiogenesis may be accompanied by a decrease in invasiveness. Thus, as the tumours are passaged through subsequent generations, they may become less diffuse and more circumscribed. The definition of the host-tumour border may increase. Other characteristics or parameters of invasiveness may also decrease, for example the expression of genes or proteins associated with invasion. A tumour or tumour cell of phenotype II, whilst still characterised as invasive, may exhibit reduced invasiveness as compared with a phenotype I tumour or tumour cell (particularly a phenotype I tumour from which it is derived).

In order to monitor progression between phenotypes I and II, the following characteristics may be studied:—invasiveness, angiogenesis and expression of stem cell markers.

Invasiveness can be studied by macroscopical or microscopical examination and inspection of the isolated tumour or tumour cells from the immunodeficient animal, particularly histological sections of the excised tumour can be taken. For example, histological haematoxylin and eosin (H&E) staining can be used on a section of excised tissue to study tissue pathology and determine invasiveness. Furthermore invasive tumour cells can be isolated from histological sections by laser capture microscopy.

Invasiveness may also be studied by imaging techniques such as MRI or PET scanning. A highly invasive tumour may show little or no contrast enhancement in an MRI scan. As invasiveness decreases, increased contrast enhancement may be seen. PET or other scans may also be used to study the definition of the host-tumour border, and how circumscribed the tumour is.

Invasiveness may also be investigated or assessed by studying the expression of genes and/or proteins associated with invasion (referred to herein as “pro-invasive” genes and/or proteins). Such proteins may include, for example, proteins which promote invasion in vivo (e.g. secreted protein and rich in cysteine (SPARC) which promotes glioma invasion in vivo), proteins which provide a substrate for migrating cells (e.g. Laminin B1 chain, Laminin B2, Laminin Gamma 1, fibronectin) or any other proteins involved in cell migration (e.g. integrins, e.g. integrin alpha 5). The expression of such genes in the tumour under investigation may be studied, for example by investigating the presence or levels of the encoded gene product or mRNA, using techniques well known in the art. Expression at the level of the protein may also be detected or assessed.

In vitro assays of the invasiveness of tumour cells may also be possible. For example the tumour cells, or a culture of the tumour cells e.g. spheroids prepared from the tumour cells, may be assessed for their ability to degrade a proteinaceous substrate, e.g. a collagen gel, for example as described for the collagen-invasion gel assay in Example 11 below.

Angiogenesis may be determined visually, since angiogenesis results in tumours with a disordered vasculature, enlarged vessels and proliferation of endothelial cells. Generally, necrotic areas are visible by MR techniques and via microscopy during angiogenesis, as the tumour secretes proteases in order to break down adjacent healthy tissue. Sections of excised tumour can be taken and various histological studies e.g. immunohistochemical staining, undertaken to allow more detailed analysis of tumour vasculature. Since various processes take place during angiogenesis a variety of markers can be used to detect various aspects of the process. During angiogenesis, some of the cells in the tumour may become hypoxic and die due to lack of blood supply. Hypoxia and dead cells may be detected as outlined below. Markers of angiogenesis can be detected, such as VEGF. Alternatively, simple observation of the vasculature may suffice.

Functional characterisation of the vasculature of excised tumours can be undertaken using injections of Indian ink. Endothelial junction morphology may be studied microscopically.

Tumours of phenotype I are angiogenesis independent and thus possess capillaries typical of normal tissue, with regular, small diameter vessels. In the intermediate phenotype and phenotype II tumours, as angiogenesis progresses, a chaotic vascular network forms, which is shown via Indian ink injections as a large and irregular area. The total vascular area (TVA) is generally significantly increased.

Sections of excised tissue can be stained with agents that bind to endothelial cell and angiogenesis dependent markers such as CD31, vascular endothelial growth factor (VEGF), Hypoxia-inducible factor 1 (HIF-1) and von Willebrand factor.

Hypoxia (a deficiency of oxygen in body tissue) may be correlated to vascular morphology, and thus angiogenesis can further be monitored using hypoxia markers such as pimonidazole. Hoechst staining can be used to test whether cells are living or dead. Live cells are capable of pumping out Hoechst, and thus only dead or apoptotic cells are labelled with Hoechst. Dead cells may indicate lack of oxygen and thus the onset of angiogenesis. Further, the integrity of basal membranes can be determined using a marker for collagen IV which is a ubiquitous component of basement membranes. Other suitable markers include BrdU (5-bromo-2-deoxyuridine) which allows DNA synthesis in (sub)populations of cells to be tracked. Thus, it may be determined whether particular cells are dividing (e.g. tumour cells, endothelial cells etc.). There are thus numerous histological methods for determining angiogenesis, and a combination of any of these methods may be used to determine whether angiogenesis is taking or has taken place.

Angiogenesis may also be assessed, as mentioned earlier, by detecting or measuring markers of angiogenesis. These may include growth factors or signalling molecules associated with angiogenesis, e.g. VEGF (e.g. VEGF-A and VEGF-C) HIF-1 and von Willebrand factor. Other angiogenic factors include platelet-derived growth factor alpha (PDGFA) and platelet-derived growth factor alpha receptor (PDGFAr), fibroblast growth factor (FGF) and fibroblast growth factor receptor (FGFr). As described earlier these may be detected histologically, e.g. by immuno- or other staining of tissue sections, or by assessing gene or protein expression of the factor concerned, which may be conducted at the protein, mRNA or gene level using well known techniques, for example nucleic acid-based assays e.g. cDNA microarrays, quantitative PCR, RT-PCR, by Western blots or immunological assays or using functional assays to assess angiogenic potential, for example by assaying for the presence or levels of the factor or factors in question in body fluids or tissues (e.g. CSF in the case of brain tumours) or in medium conditioned by culture of the tumour cells (e.g. spheroid culture). An aortic ring assay for endothelial sprouting is described in Example 11 below.

As described further in the Examples below, an angiogenesis-independent phenotype may be identified, or characterised, by dividing cells between blood vessels with no Hoechst leakage into the surrounding parenchyma. This indicates normal vasculature among dividing tumour cells.

An angiogenesis-dependent phenotype may be manifested, as described above, by tumours with disordered vasculature, e.g. irregular vessels, enlarged or dilated vessels, endothelial cell proliferation, necrotic and/or hypoxic regions in the tumours, and Hoechst leakage into the surrounding parenchyma. Expression of angiogenesis-associated factors (e.g. angiogenesis-promoting or angiogenesis-signalling factors), for example VEGF will be detected, or may be increased, as compared with an angiogenesis-independent tumour.

Scans of the live animal implanted with the tumour cells may also be useful in determining invasiveness and angiogenesis. MRI (magnetic resonance imaging) and PET (Positron Emission Tomography) scans can be used, with suitable markers as appropriate, contrast agents (MRI) and radio-labelled thymidine (PET), collagen IV labelling and BrdU (immunohistochemistry).

Thus invasiveness and angiogenesis can be detected and monitored using methods routine in the art.

Preferably, however, the progression from phenotype I to phenotype II cells is monitored by performing flow cytometric DNA analysis of the excised tumour. The DNA ploidy (DNA content) of the tumour cells changes during the progression. Phenotype I cells have a diploid DNA content which gradually changes to an aneuploid content during passaging. A small population of the cells may have an euploid content, generally about 10%.

Alternatively or additionally, the cells can be tested for expression of stem cell markers such as Nestin, CD133, Vimentin and Musashi (an RNA-binding protein involved in assymetric cell division in neural development (Okabe et al., 2001, Nature 411, 94-98), since these are lost during progression to cells of phenotype II.

The migratory pattern or behaviour of the tumour cells may also be studied, for example by histological investigation, e.g. of tumour cell distribution. The ability of the cells to grow in stem cell media may be investigated.

In particular, as described further in the Examples below, the process of progression from phenotype I to phenotype II may be characterised by a reduction in stem cell markers. The results reported below further show that pro-invasive genes may be up-regulated, and angiogenesis signalling genes down-regulated in tumours of phenotype I. In contrast, pro-invasive genes may be down-regulated in the angiogenesis-dependent tumours of phenotype II derived therefrom. Angiogenic factors may be up-regulated. Thus, the transition from angiogenesis-independent growth to angiogenesis-dependency may be characterised by a down-regulation of pro-invasive genes and a loss of stem cell markers.

The generation of tumours or tumour cells of phenotype II from tumours or cells of phenotype I forms a further aspect of the present invention. Accordingly, in a further aspect, the present invention provides a method of generating cells of a defined phenotype, being invasive and angiogenesis-dependent (phenotype II), from a tumour sample, said method comprising the steps of:

(i) culturing tumour cells from said tumour sample in order to establish multicellular spheroids;

(ii) implanting said multicellular spheroids into an immunodeficient animal;

(iii) allowing a tumour to develop, in the case of this first implantation step, said tumour being invasive and angiogenesis-independent (phenotype I);

(iv) isolating a tumour sample or tumour cells from said animal (e.g. by optionally sacrificing said animal, optionally removing the tumour, and isolating tumour cells derived from said multicellular spheroids, from said animal or from said tumour);

(v) repeating steps (i) to (iv) until the tumour becomes angiogenesis-dependent.

In a further aspect of the invention, cells of phenotype II may be isolated from a tumour thus obtained by a method analogous to that described earlier for phenotype I, using the animal or the tumour containing or having a tumour phenotype of type II.

Thus, this aspect of the invention provides a method of isolating cells of a defined phenotype, being invasive and angiogenesis-dependant (phenotype II) from a tumour sample, said method comprising the steps of:

(i) culturing tumour cells from said tumour sample in order to establish multicellular spheroids;

(ii) implanting said multicellular spheroids into an immunodeficient animal;

(iii) allowing a tumour to develop, in the case of this first implantation step, said tumour being invasive and angiogenesis-independent (phenotype I);

(iv) isolating a tumour sample or tumour cells from said animal (e.g. by optionally sacrificing said animal, optionally removing the tumour, and isolating tumour cells derived from said multicellular spheroids, from said animal or from said tumour);

(v) repeating steps (i) to (iv) until the tumour becomes angiogenesis-dependent; and

(vi) isolating tumour cells of said phenotype from said animal.

Cells of phenotype II isolated, or obtainable, according to such methods from a further aspect of the present invention.

The method of this aspect of the invention for generating cells of phenotype II may also be viewed as a method of generating a phenotype II tumour, or an animal model of a phenotype II tumour, analogously as described for the phenotype I tumour/animal model above.

The present invention thus also provides an animal model which is obtainable by the above-mentioned method.

The animal model is thus prepared as described above, by serially transplanting tumour spheroids into animals. The animal model can be selected at any point in the progression between tumour cells of phenotype I and cells of phenotype II, and the animal models will thus be useful tools in analysing the progression of tumour cells in vivo from one phenotype to another.

Accordingly, the present invention provides a method for generating an animal model with a tumour of phenotype II, or an intermediate or mixed phenotype between phenotype I and phenotype II, from a tumour sample, said method comprising:

(i) culturing tumour cells from said tumour sample in order to obtain multicellular spheroids;

(ii) implanting said spheroids into an immunodeficient animal;

(iii) allowing a tumour to develop, in the case of this first implantation step, said tumour being invasive and angiogenesis-independent (phenotype I);

(iv) isolating a tumour sample or tumour cells from said animal (e.g. by optionally sacrificing said animal, optionally removing the tumour, and isolating tumour cells derived from said multicellular spheroids, from said animal or from said tumour).

(v) repeating steps (i) to (iv) one or more times wherein to obtain an animal model of phenotype II, said steps are repeated until the tumour becomes angiogenesis-dependant.

Although it is preferred to use the method of the invention as described above for the generation of cells of phenotype I as the preliminary first step in the above-mentioned methods for generating phenotype II tumours or cells, or animal models thereof, (i.e. a method involving a tumour cell culture step of up to 9 days to obtain spheroids for implantation), this is not absolutely necessary, and if desired longer spheroid culture periods may be used, as described above (e.g. up to 21 days etc.), in the initial or primary tumour generation step, i.e. the step of generating a type I tumour.

Thus in preferred embodiments, the methods of the invention for generating and/or isolating phenotype II tumour cells or animal models comprise:

(i) generating a tumour of phenotype I as hereinbefore described;

(ii) isolating tumour cells therefrom;

(iii) culturing said tumour cells of phenotype I to obtain multi-cellular spheroids;

(iv) implanting said spheroids into an immunodeficient animal;

(v) allowing a tumour to develop in said animal.

Optionally steps (ii) to (v) involving tumour cell isolation from a generated tumour, spheroid implantation, and tumour development are repeated one or more times, in the case of obtaining a phenotype II tumour or model, until the tumour becomes angiogenesis-dependant.

The method of generating tumours or cells of phenotype II, whether for isolation or for retention in an animal model, may involve monitoring the tumour in vivo or ex vivo for signs of angiogenesis development. Cells and tumours of phenotype II are angiogenesis dependent and are thus highly vascularised. Any suitable means may be used to monitor vessel formation in the tumour, including MRI and PET scans in vivo and histology staining of sections in vitro using stains such as Pimonidazole, angiogenesis and endothelial cell markers and Hoechst stain, as described above.

The number of transplantation passages that are required to generate tumours or cells of phenotype II from tumours or cells of phenotype I varies according to the tumour tissue type and the organ into which the transplantation occurs. Generally, tumours of phenotype II cells are obtained within 1 to 10 transfers of the cells of phenotype I into an animal. Thus, 1 to 10 serial transplants are made, more preferably 1 to 7, 2 to 6, 3 to 6, 2 to 5, 3 to 5 or 4 to 6 serial transplants are made. In the case of brain tumours, it has been found that, for example, tumours of phenotype II are established in 5 generations. Thus, to establish brain tumours of phenotype II, 5 serial transfers may be made.

The tumour cells are cultured as multicellular spheroids between each transplantation step. The method of culturing cells in order to obtain multicellular spheroids is discussed previously, and any suitable method may be used. The cells may be maintained as multicellular spheroids in culture for any suitable length of time prior to transplantation into the immunodeficient animal. Preferably, the cells are cultured as multicellular spheroids for 1 day to 6 weeks, more preferably 1 day to 3 weeks, most preferably up to 10, 9 or 7 days, e.g. up to one week. When culturing tumour cells in the appropriate conditions to form multicellular spheroids, spheroid formation may take 3 to 5 days in culture (Bjerkvig et al., supra). Once obtained, the spheroids are then maintained in culture as described above. The total post-extraction culturing period is thus about 3 days to 6 weeks, preferably 3 days to 3 weeks, most preferably 3 days to 10 days, e.g. 3-9 or 3-7 days. Spheroids may survive in culture for over 10 weeks, and thus any suitable culture time may be used between transplantation events. However, it is preferred that the cells are cultured for 3 to 10 days post-excision in the appropriate conditions in order to obtain multicellular spheroids.

The technique for culturing spheroids is as described previously, and any suitable method of culturing the cells may be used.

The spheroids are implanted into immunodeficient animals as described earlier. Thus, the immunodeficient animal is preferably a mouse or rat, and the spheroids are implanted at any suitable location, preferably orthotopically transplanted.

The cells of phenotype II are preferably isolated from the immunodeficient animal, to allow characterisation and further examination of the properties of the cells. The isolation step thus forms a preferred additional step in the generation of cells of phenotype II, as described generally above. The immunodeficient animal carrying cells of phenotype II (which can be detected as described previously) may be sacrificed (e.g. by CO₂ inhalation or other suitable means) when signs of tumour-development appear (e.g. clumsiness in the case of brain tumours). The organ or tissue containing the tumour is excised. The tumour cells can then be separated from the animal cells using flow-cytometric cell sorting techniques or magnetic bead separation techniques as described previously, or any known or desired technique. Prior to separation of the tumour-derived cells from the animal cells, it is preferred to dissociate the tumour cells into a cell-suspension. Preferably, the dissociation of the cells takes place via enzymatic means, but mechanical methods are also envisaged. The cells can thus be isolated using antibodies that bind to cells of a particular origin (i.e. human cells using pan-anti-human antibodies). A substantially pure, or substantially homogenous (as defined above with respect to cell content) preparation of cells of phenotype II may thus be obtained, and represents a further aspect of the invention.

The present invention further relates to the production, isolation or generation of cells of phenotype III from a tumour. Tumours and tumour cells of phenotype III as defined herein are non-invasive and angiogenesis dependent. By “non-invasive” is meant that the tumour, or the tumour from which the cells derive, is not able to invade or infiltrate surrounding cells or tissue. The tumour thus grows as a discrete or localised or circumscribed lesion. The host-tumour border may be well-defined. As discussed above, this may be readily determined using standard techniques (e.g. morphological and macro- and microscopic inspection techniques as described above). Such cells exhibit or express significantly fewer stem cell markers than cells of phenotype I.

In order to obtain cells of phenotype III from a tumour, said tumour is cultured in order to obtain multicellular spheroids using the techniques as described previously. However, an important difference is the length of time the tumour cells are cultured in vitro prior to implantation. The multicellular spheroids are maintained in culture for 5 to 10 weeks, preferably 5 to 7 weeks, most preferably about 6 weeks (e.g. post-sampling or from first placing the tumour cell into culture). It will be understood that the culture medium will be changed as necessary in order to maintain the cells. The multicellular spheroids thus obtained are implanted into an immunodeficient animal and a tumour is allowed to develop, as described previously. Thus for example, the animal may be monitored until signs of disease (tumour growth) are apparent, and thus a tumour containing cells of phenotype III has developed, or the animal is simply maintained until a suitable time interval to allow for tumour development has passed. Thus, an animal containing a tumour or cells of phenotype III may be developed, and the “animal model” containing the tumour forms a further aspect of the invention. The tumour or cells of phenotype III can be isolated from the animal to permit further study on these cells. Thus, either the animal model or the isolated cells may be the subject of further investigation and experimentation.

In a further aspect, the present invention thus provides a method of generating cells of a defined tumour phenotype, being non-invasive and angiogenesis-dependent (phenotype III) from a tumour sample, said method comprising the steps of culturing tumour cells of said tumour sample for 5 to 10 weeks in order to establish multicellular spheroids, and implanting said multicellular spheroids thus obtained into an immunodeficient animal.

The above-mentioned method results in an animal with implanted tumour cells, from which a tumour may develop. Thus the implanted spheroids are allowed to develop into a tumour. The present invention thus extends to a method of generating a tumour of phenotype III and to the animal model thus derived.

Therefore, in a related aspect, the present invention also provides a method of generating an animal model of a defined tumour phenotype, being non-invasive and angiogenesis-dependent (phenotype III tumour) from a tumour sample, said method comprising the steps of culturing said tumour sample for 5 to 10 weeks in order to establish multicellular spheroids, and implanting said multicellular spheroids thus obtained into an immunodeficient animal.

The invention further extends to an animal model obtainable by the method of the invention.

Tumour cells of phenotype III may be isolated from the animal by standard methods, as discussed above, for example by removing or excising the tumour from the animal and isolating the cells therefrom, or by directly isolating the cells from the animal or the animal tissue or organ in which the tumour has developed.

Thus, in a further aspect, the present invention also provides a method of isolating cells of a defined tumour phenotype, being non-invasive and angiogenesis-dependent (phenotype III) from a tumour sample, said method comprising the steps of culturing tumour cells of said tumour sample for 5 to 10 weeks in order to establish multicellular spheroids, implanting said multicellular spheroids thus obtained into a laboratory animal, and isolating tumour cells of said phenotype from said animal.

Cells of phenotype III, isolated, or obtainable, by the above-described methods form a further aspect of the invention.

The method of isolating cells of phenotype III can comprise the following steps:

allowing a tumour to develop in said animal (e.g. by monitoring said animal until symptoms of disease tumour presence occur or by simply maintaining said animal for a time period suitable to allow a tumour to develop), optionally sacrificing the animal and isolating tumour tissue or cells therefrom.

The cells may be isolated as discussed previously.

It forms a preferred aspect of this invention that at least two cell phenotypes are isolated from the same tumour sample (i.e. I and II, I and III or II and III). More preferably, cells of all three phenotypes (I, II and III) are isolated or generated from the same tumour sample. Cells thus obtained are of great clinical interest since it will be possible to directly compare cells of the different phenotypes which have the same origin. The tumour growth and progression can thus be elucidated, together with changes in gene expression, and resistance to chemotherapeutic and radiotherapeutic agents and morphological characteristics may be studied etc. Such cells represent a unique tool for identifying new targets for therapy, for example.

Thus, in a preferred aspect the invention provides a method for generating cells of phenotypes I, II and III from a tumour sample, said method comprising the steps of culturing tumour cells of said tumour sample in order to obtain multicellular spheroids, wherein (a) to obtain cells of phenotype I the tumour cells are cultured for up to 21 days, and wherein (b) to obtain cells of phenotype III, the cells are cultured for 5 to 10 weeks, and implanting said multicellular spheroids into an immunodeficient animal, and wherein (c) to obtain cells of phenotype II, the method comprises the steps of (i) isolating cells of phenotype I from said animal, (ii) culturing said cells in order to obtain multicellular spheroids; (iii) implanting said multicellular spheroids into an immunodeficient laboratory animal; (iv) allowing a tumour to develop in said animal; (v) isolating tumour cells from said animal (i.e. tumour cells derived from said multicellular spheroids); (vi) culturing the tumour cells in order to obtain multicellular spheroids, and (vii) repeating steps (iii) to (vi) until the tumour implanted into said animal becomes angiogenesis-dependent.

The above-mentioned method thus results in the generation of three animals containing tumours of a particular phenotype. The animals may be studied per se, or sacrificed and/or the cells of interest isolated as outlined previously. As set out above for previous aspects of the invention, this aspect thus also includes a method of generating animal models of all three phenotypes, and a method for isolating cells of all three phenotypes, according to analogous steps and principles.

In the method of invention, any suitable tumour (e.g. solid tumour) may be sampled and used to generate or isolate the cells of the different phenotypes. However, the present inventors have found that the present method is particularly applicable to brain tumours, and thus it is preferred that the tumour sample is a sample of a brain tumour. All types of brain tumour can be used in the method of the invention, for example gliomas (tumours derived from neuroglial cells) and medulloblastomas. There are 3 main types of glioma; Astrocytoma, Ependymoma and Oligodendroglioma, differing in the cell of origin. Brain tumours are classified into grades (1 to 4) according to how fast they are likely to grow. Low grade gliomas (grade 1 and 2) are the slowest growing brain tumours. All grades of tumour are suitable for use in the method of the invention. Astrocytoma grades 3 and 4 may also be called Anaplastic Astrocytoma and Glioblastoma Multiforme, respectively. These types of brain tumour are the most common in adults. Further, some gliomas may be a mixture of 2 or even 3 of the different types of glioma. Any such tumour may be used in the method of the invention.

If the tumour biopsy sample is derived from brain tissue, it is preferred that the multicellular spheroids derived therefrom are implanted into the brain of an immunodeficient laboratory animal, i.e. the transplantation is orthotopic.

The tumour biopsy is preferably taken from a human patient. The tumour is fragmented into small pieces immediately after excision, usually within 20 minutes of excision, and cultured in order to obtain multicellular spheroids. Such steps are as described previously. Cells of all phenotypes (I, II and III) may be obtained from the tumour biopsy sample.

When brain tumour tissue is used in the method of the invention, it will be understood that the stem cell markers which can be detected in order to check cells of phenotype I have been obtained and to monitor the progression between cells of phenotype I and phenotype II will be brain-cell specific. Thus, suitable brain stem cell markers include Nestin, vimentin, Musashi, NG-2 proteoglycan, PSA-NCAM (neural cell adhesion molecule), CD-133, Tuj-1 (class III tibulin) 3′6′-isoLD1 and 3′-isoLM1. Nestin and vimentin are intermediate filament proteins, and are expressed by neural stem cells (Dahlstrand et al., Cancer Res., 1992, 52(19), 5334-41 and Salinen et al., Cancer Res., 2000, 60(23), 6617-6622). NG2 proteoglycan is expressed during embryogenesis and is especially associated with brain capillaries. NG2 is expressed during a period of rapid expansion of the brain vasculature and is down regulated as the vessels terminally differentiate. In the adult central nervous system (CNS) oligodendroglial precursor cells are known to express NG2. The present inventors have recently shown that overexpression of NG2 increases tumour initiation and growth rates, neovascularisation and cellular proliferation, which predisposes to a poorer survival outcome (Cheya et al., Faseb J., 20002, 16(6) 586-588). 3′-iso-LM1 and 3′6′ isoLD1 are gangliosides which are expressed in relatively large amounts in brain areas invaded by brain tumours (Wilkstrand et al., Prog Brain Res., 1994, 101, 213-23). These gangliosides are not expressed in normal adult brain (after 2 years of age) but are found during brain development.

Pancreatic tumours also form a preferred tumour to be biopsied and cultured using the method of the invention. Pancreatic tumours may be excised and cultured as multicellular spheroids as discussed previously. The spheroids thus obtained may be implanted into the pancreas of an immunodeficient laboratory animal (i.e. orthotopic transplantation), but it is preferred that the spheroids are transplanted into the brain or liver, or any suitable highly vascularised tissue. Pancreatic tissue is loose and not ideal for transplantation. With regard to pancreatic tumours, pancreatic stems cells may express the stem cell markers nestin, k20, vimentin and bcl-2, amongst others.

As defined above, in one aspect, the method of the invention provides a method for isolating or generating cells of phenotypes I, II and/or III from a single tumour sample. The method presented here is the first method demonstrated reliably to isolate or generate cells of all three phenotypes using the step of implanting multicellular spheroids into an immunodeficient animal. These cells of the various phenotypes have been classified and characterised by the inventors of the present application for the first time. Thus, the isolated cells form a further aspect of this invention.

The present invention thus provides a substantially homogenous preparation of tumour cells of phenotype I, wherein said cells are invasive and angiogenesis independent.

The invasive and angiogenesis characteristics may be determined as outlined previously. The isolated cells may be used as a tool in the identification of novel genes and in the search for new chemotherapeutic agents which are effective against the transformed stem cell population (cells of phenotype I). This is thought by the inventors of the present application to be particularly advantageous, since the cells of phenotype I are thought to represent the initial transformed stem cells from which more differentiated cells in tumours derive. It is thought that the initial transformation event that converts a normal cell into a tumour or cancerous cell occurs in the stem cell population. The transformed stem cells are thought to represent a self-renewing cell population which gives rise also to more differentiated (i.e. non-stem cell) tumour cells (e.g cells of phenotype II or phenotype III). Such cells are of particular interest in the field since the present inventors have found that cells of phenotype I are more resistant to chemotherapy and radiotherapy than cells which have lost their stem-cell characteristics. Therefore, preparation of isolated, substantially homogenous cells of phenotype I is an important tool in the study of agents that can successfully target and destroy this population of cells.

The term “Substantially homogenous” is defined above, and for example means that at least 75% of the cells are of the defined phenotype, preferably 80-100%, more preferably 90-100%, e.g. at least 90, 91, 92, 93, 94, 95, 96, 97, 98 or 99% of the cells present are of the defined phenotype.

The present invention further extends to a substantially homogenous preparation of tumour cells of phenotype II, wherein said cells are invasive and angiogenesis-dependent.

Additionally, the present invention provides a substantially homogenous preparation of tumour cells of phenotype III, wherein said cells are non-invasive and angiogenesis-dependent.

Cells of phenotypes II and III can be studied in an analogous way to cells of phenotype I. Cells of phenotypes II and III are thought to be derived from the transformed stem cells, and have lost the ability to self-renew and have lost the majority of stem-cell markers. Thus, these cells are also of interest to researchers since they represent particular cells types from the heterogeneous tumour cell that will need to be ablated in successful cancer therapy.

The cells of phenotypes I, II and III are obtainable by the methods as hereinbefore defined.

In a preferred aspect of the invention, cells of the three phenotypes (I, II and III) are derived from a single tumour sample. The three types of cell thus derived provides a unique tool for the study of the progression of the tumour cells, and allows a comparison to be made between the “progenitor” transformed stem cells (i.e. cells of phenotype I) and “descendant” tumour cells which have lost the stem-cell characteristics, for example a comparison of gene and/or protein expression.

The use of cells of phenotypes I, II and III in determining gene expression patterns, drug sensitivity testing, determining new targets for therapy and determining biological characteristics of a tumour are envisaged. The present invention thus extends to the use of cells of phenotypes I, II and/or III in determining differential gene expression, or determining differentially expressed proteins.

In order to use the cells to determine differential gene expression, the mRNA is extracted from isolated cells of phenotype I, II and III. Differential gene expression can then be determined using standard cDNA microchip technology, differential display technology or Serial Analysis of Gene Expression technology. These are well known technologies in the art.

To look for differentially expressed proteins, the 2-D gel electrophoresis blots from proteins extracted from cells of phenotypes I, II and III may be compared. Alternatively, differentially expressed proteins can be detected by chromatography techniques such as HPLC or FPLC.

Thus, the present invention enables cells or tumours of two or more of the three different phenotypes to be prepared and compared, for example between each other and/or to normal (non-tumour) cells (e.g. stem cells or differentiated tissue cells). Advantageously, genomic and/or proteomic profiles of the different phenotypes may be compared. To enable comparative genomic and/or proteomic profiling to be performed, genomic and/or protein libraries may be prepared from each of the different phenotypes (or from two different phenotypes being compared). By comparing such libraries, genes and/or gene products, or expression profiles unique to, or that characterise the respective phenotypes may be identified. Such genes or gene products etc. may represent novel targets for therapy. Thus, by way of example, comparison of the gene and/or protein expression profiles may be carried out, e.g. between transformed stem cells and non-transformed normal stem cells from the same tissue, which may enable the identification of molecular events, and hence potential therapeutic targets, leading to tumour initiation. Comparison of phenotypes I and II may enable the identification of molecular events, and hence targets, determining tumour progression (e.g. angiogenesis). Comparison of phenotype III with phenotype I and/or II may identify molecular events, and hence targets, responsible for tumour invasion. Techniques for genomic and/or proteomic expression and profiling and comparison etc. are widely described in the literature.

Thus cells of phenotype II and/or III can thus be compared to cells of phenotype I with regard to several characteristics, allowing new targets for therapy to be identified.

The present invention provides the use of tumour cells of phenotypes I, II and/or III to identify therapeutic targets.

The invention will now be described in more detail, in the following non-limiting Examples, with reference to the drawings in which:

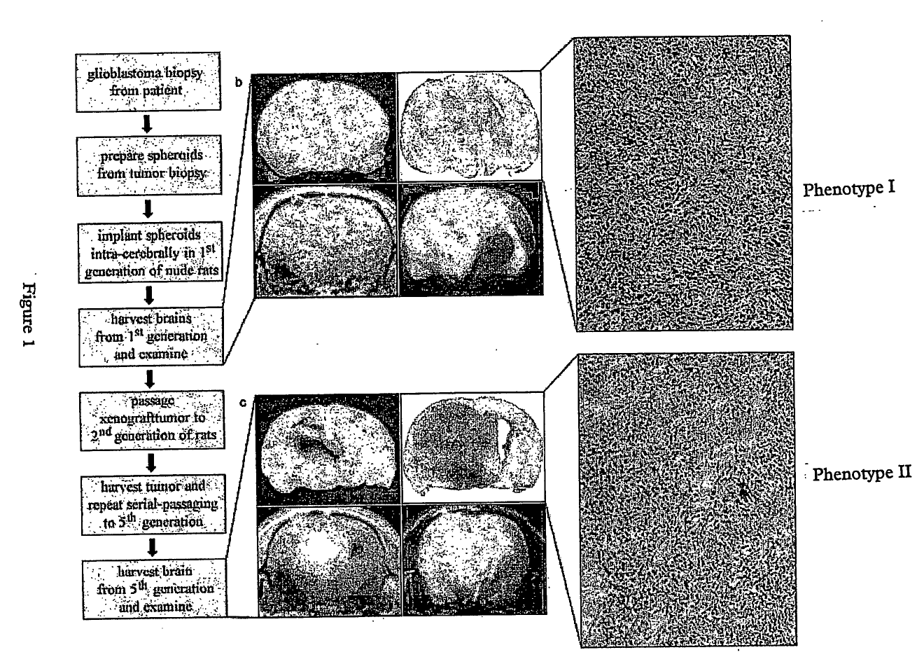

FIG. 1 shows a schematic representation of a particular embodiment of the invention, namely generation of tumours of phenotype II from a tumour sample (glioblastoma). Panel b shows the macroscopic appearance of phenotype I tumours (upper left), a histological section of the same tumour (upper right). In theory, a T1 contrast enhanced MRI indicating no contrast enhancement demonstrates that no angiogenesis is taking place. The T1 image in the lower panel c left were severe contrast enhancement is achieved and thus angiogenesis is demonstrated. The MRI scans show two diffusely invasive growing tumours;

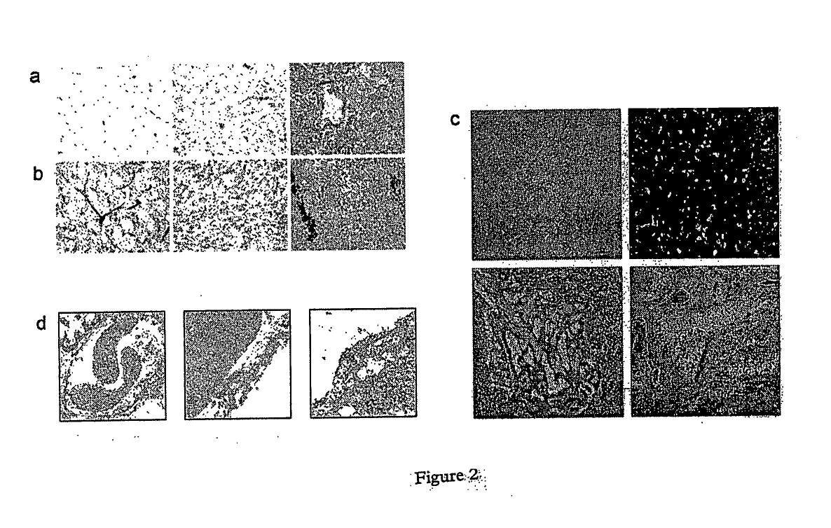

FIG. 2 shows results obtained after histological staining or analysis of tumours obtained in the Examples of the present application; FIG. 2 (a) left panel: normal blood vessels stained for CD31, middle panel CD31 staining phenotype I tumour; right panel: CD31 staining phenotype III tumour; (b) the same vasculature as panel (a) observed after Indian ink injection; (c) upper panels phenotype I tumour stained for collagen IV, Hoechst and hypoxia, indicating mature blood vessels in phenotype I tumours, but not in 5th generation tumours (phenotype II); (d) transmission electron microscopy showing mature endothelial cells in phenotype I tumours (left and middle panel) but not in phenotype II tumours;

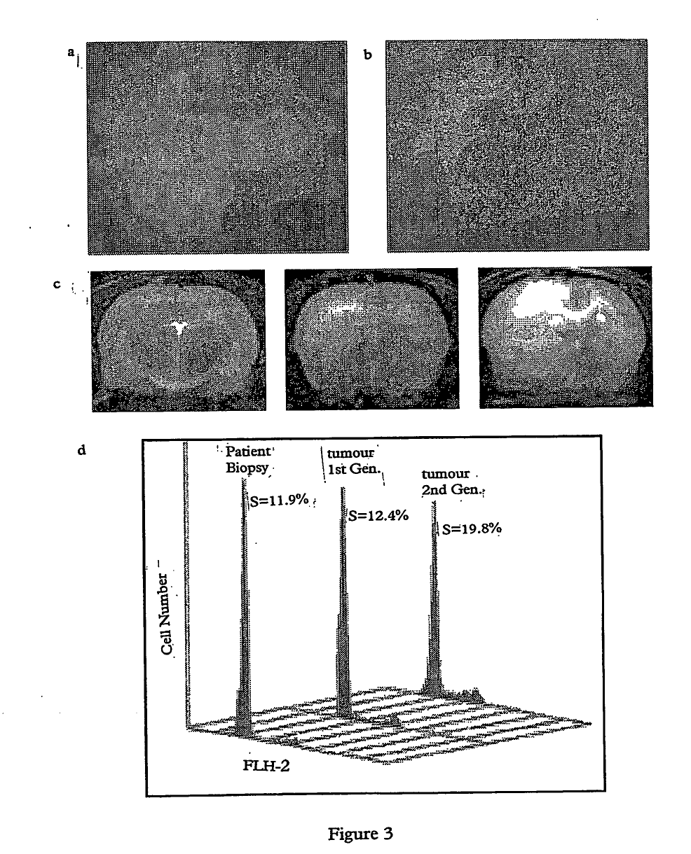

FIG. 3 shows detection of cell growth and division in the non-angiogenic tumour phenotype. This is demonstrated in several ways—FIG. 3(a) shows FLT-pet results. The scan shows a diffuse uptake of radio-labelled thymidine, indicating a disseminated spread of tumour cells; FIG. 3(b) shows BrdU labelling results. Dividing cells are shown to be spreading locally from the injection site as well as invading along the corpus callossium to the contralateral hemisphere; FIG. 3(c) shows MR-scans at three different time points in order to study tumour growth over time. The scans display diffusely growing lesions, accompanied by progressive oedema, causing a shift of midline structures in the latter stages; and FIG. 3(d) shows flow cytometric cell cycle distribution curves. FACs confirmed the presence of mitotic cells in the primary sample as well as the tumours from the different generations. The tumour is of phenotype I;

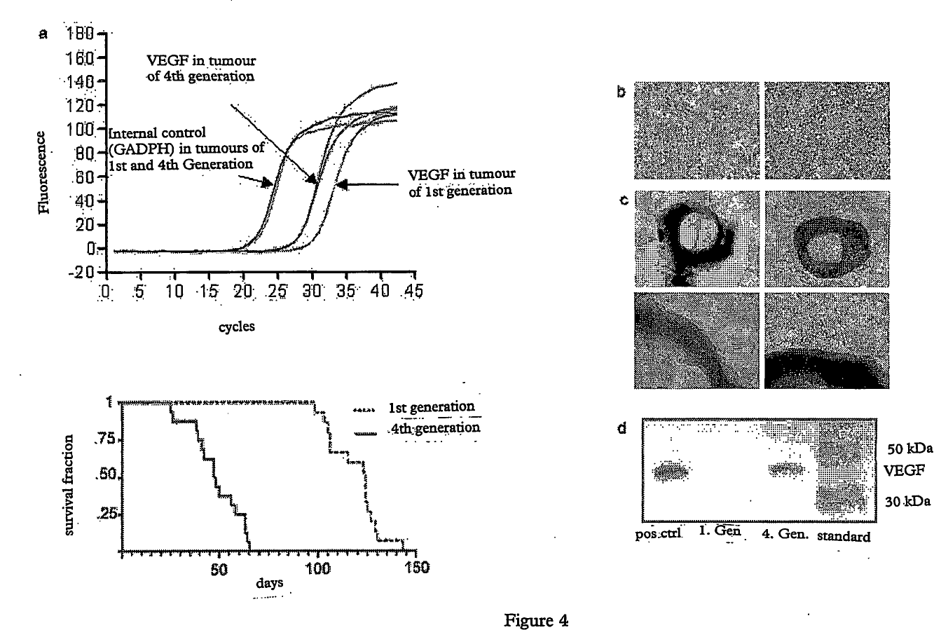

FIG. 4 demonstrates that phenotype I tumours do not secrete angiogenic factors whilst tumours derived from later generations do (4th and 5th generation e.g. mixed or phenotype II tumours); FIG. 4a shows real time PCR results from VEGF; FIG. 4b shows immunohistochemistry of phenotype I tumours and of phenotype II tumours showing strong positive results in the phenotype II tumours thus confirming the PCR results; FIG. 4c shows aortic ring assay results showing neovascularization when the aortic ring is exposed to conditioned medium from spheroids derived from 5th generation tumours; FIG. 4d shows detection of VEGF in the cerebrospinal fluid of rats bearing 5th generation tumours. Shown are also Kaplan Myer curves of rats bearing phenotype I tumours and rats bearing phenotype II tumours;

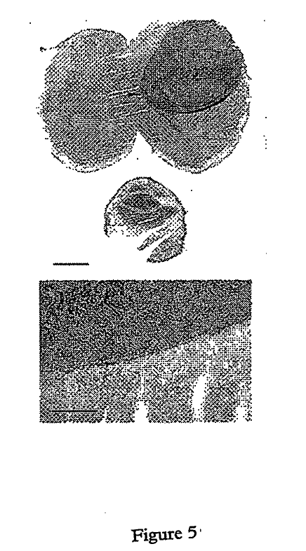

FIG. 5 shows a non-invasive glioma derived from a human biopsy spheroid, maintained in culture for six weeks and then transplanted into a nude rat brain. The tumour is negative for the stem cell marker nestin and show contrast enhancement on MRI scans indicating that the tumour depend on angiogenesis for growth. It is thus a tumour of phenotype III as defined herein;

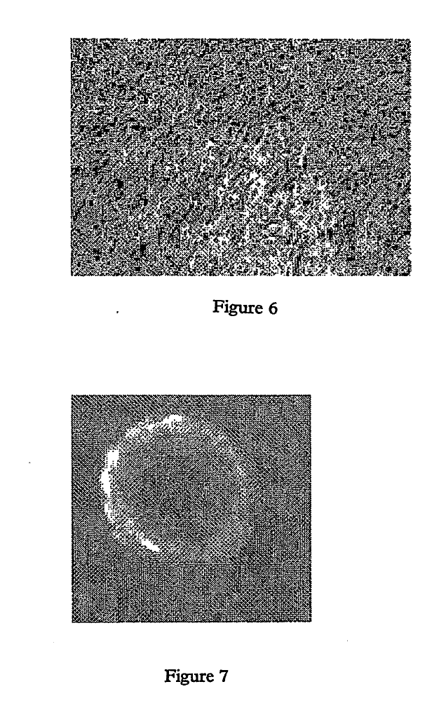

FIG. 6 shows the results obtained after immunostaining a section of brain tumour for the stem cell marker nestin in a phenotype I tumour;

FIG. 7 shows a transformed “neurosphere” isolated from phenotype I tumour grown in stem cell medium. These cells represent a lineage restricted cell type within the brain tumour;

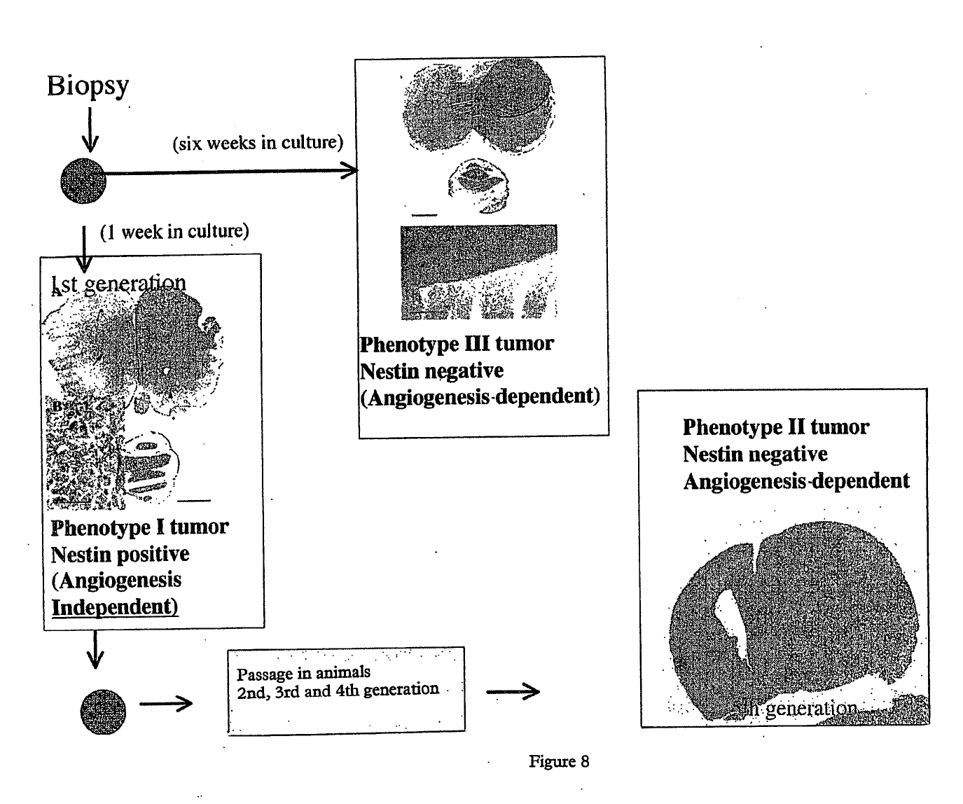

FIG. 8 is a schematic representation of the technique for isolating or generating different tumour phenotypes (Phenotype I, Phenotype II and Phenotype III) derived from a single brain tumour biopsy;

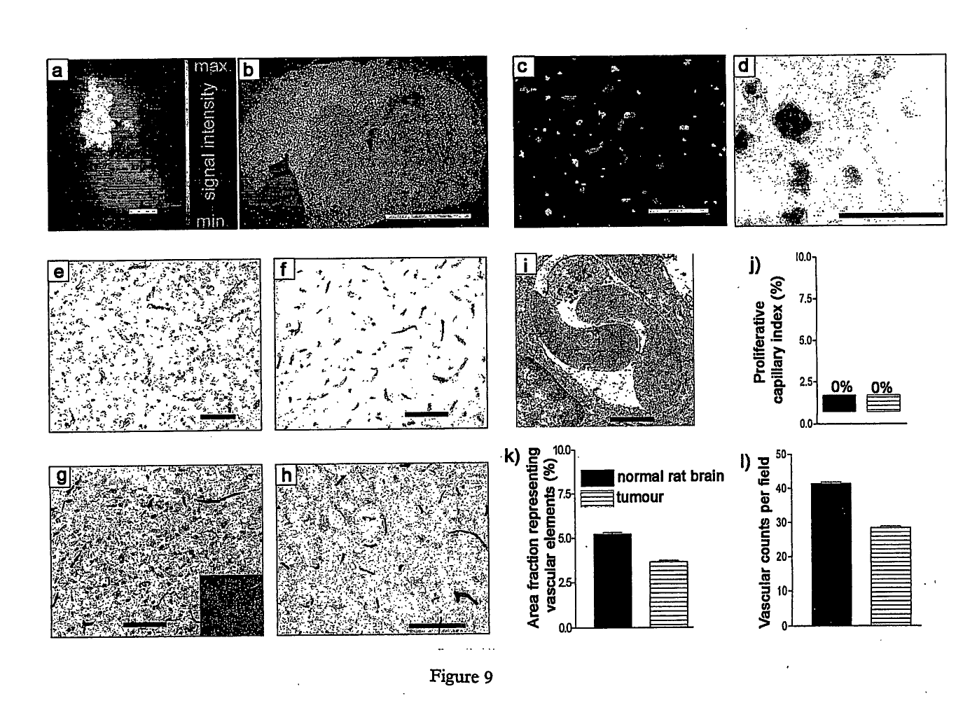

FIG. 9 shows tumour growth without angiogenesis;

FIG. 9(a) shows a PET-scan showing a horizontal rat brain section with a tumour after [¹⁸F]FLT injection. Signals of varying intensities are seen throughout the brain, indicating an extensive spread of dividing tumour cells; (b) shows coronary rat brain section co-stained with BrdU (green) and Collagen IV (red). Dividing cells are seen spreading along the corpus callossum; (c) shows triple-staining for BrdU (green), Collagen IV (red) and Hoechst (blue) demonstrates dividing tumour cells infiltrating the vascular network, without leakage of Hoechst; (d) shows co-staining for CD31 (red) and Ki67 (brown) show several Ki67 positive tumour cells while the endothelial cells were uniformly negative; (e) shows CD31-staining of vessels in the tumours; (f) shows the normal brain. Arterial injection of indian ink; (g) shows in the tumour, and (h) shows in normal brain. Pimonidazol-staining (green) show no hypoxia in the tumour (g-inserted); (i) shows TEM-picture of a tumour blood vessel displaying a well defined basal lamina with tight junctions; and (j-l) show morphometric quantification of vascular parameters in the tumour as well as in the normal brain. All bars 100 μm. The experimental methods are described in Example 11;

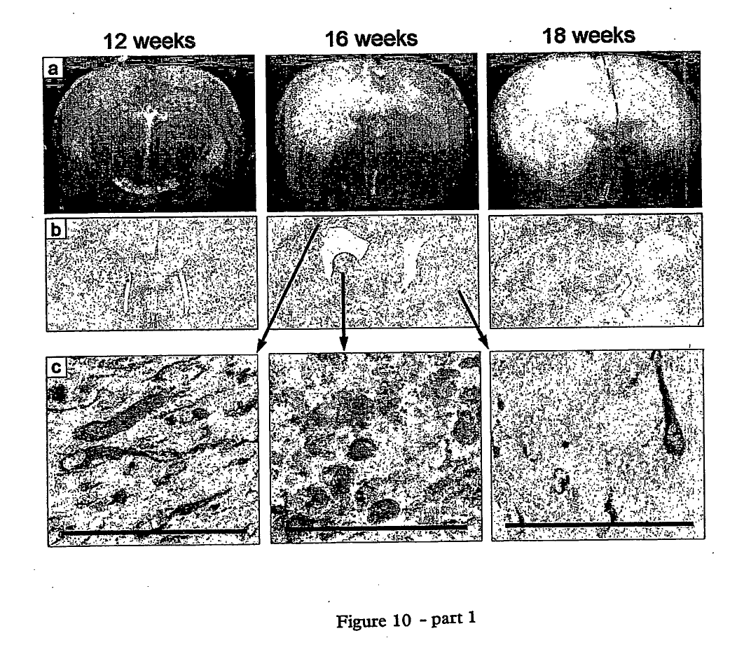

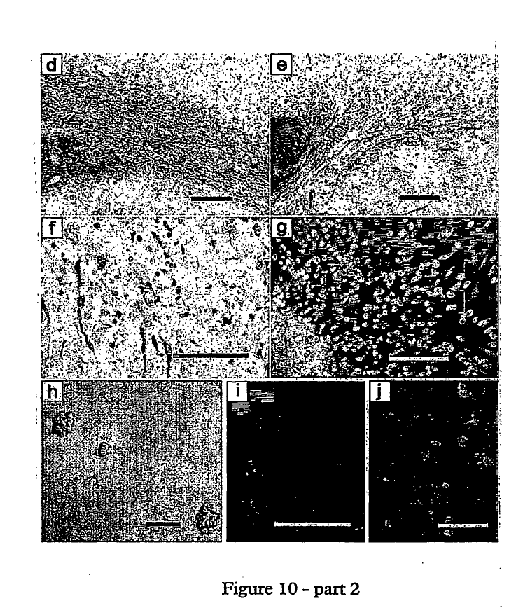

FIG. 10 shows spatio temporal distribution of cancer stem cell growth; FIG. 10(a) shows repeated MRI-scans (T₂-sequence) of the same rat at three different time points show a poorly circumscribed lesion that extends along the corpus callossum and occupies both hemispheres in the terminal stage. A shift of the midline structures (dotted lines) indicates an expanding lesion; (b) on corresponding brain sections, the main tumour mass has a purple color due to immunostaining with a human-specific antibody against the neural stem cell marker vimentin. The lower panels show co-staining with vimentin (red) and Ki67 (brown); (c) dividing and non-dividing tumour cells are seen in all regions of the brain; corpus callossum (left), tumour bulk (middle) and contralateral hemisphere (right). (d) migration along corpus callossum of nestin-positive cancer cells from a tumour spheroid; (e) human neural stem cells. Both the tumour and the normal stem cells were implanted in the right hemisphere; (f) Nestin-positive cancer cells invading the parenchyma; (g) Mushashi-positive cells (green) migrating from a tumour spheroid (red) cultured on a plastic substrate; (h) light microscopy showing spheroid formation by cancer cells grown in stem cell medium; (i) mid-section of a tumour spheroid cultured in stem cell medium, stained with a live-dead kit and DAPI showing viable cells; and (j) BrdU-staining (red) of cancer cells growing in stem cell medium. All bars 100 μm;

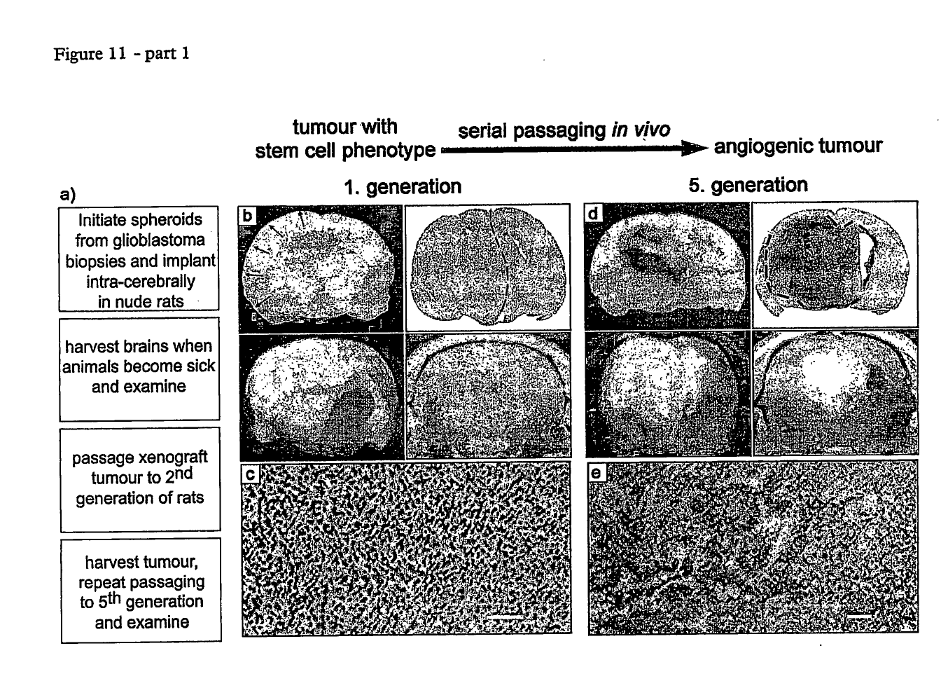

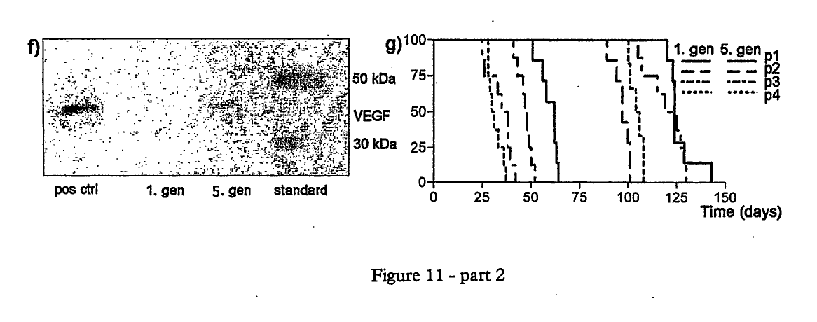

FIG. 11 shows angiogenesis-independent stem cell tumours, can give rise to angiogenesis-dependent brain tumours; FIG. 11(a) shows experimental design: Tumors were serially passaged for five generations in nude rats; (b) coronary rat brain sections of 1st generation tumours: A moderate enlargement of the hemisphere (black arrows) causing a shift of the midline structures away from the implantation site (dotted lines), reveal the presence of an expansive lesion upon gross macroscopic inspection and H/E-staining (upper panel). T₂-weighted MRI scan show an increased signal in the right hemisphere (lower panel, left), with no signs of contrast enhancement (lower panel, right); (c) no pathological vasculature or necrosis is seen, even in highly cellular areas in the tumour at high magnification; (d) several necrotic areas are recognised macroscopically in 5th generation tumours (upper left), which appear more circumscribed (dotted lines) with numerous enlarged vessels (upper right). T₂-weighted MRI show an increased signal intensity (lower left), and a strong contrast enhancement on T₁-weighted images (lower right) (e) at high magnification, necrotic areas and irregular vessels are seen in the 5th generation tumours; (f) Western-blots show the presence of VEGF only in the cerebrospinal fluid from animals with 5th generation tumours; (g) Kaplan Meyer curves showing angiogenesis to coincide with a decreased median survival from 113 to 43 days in the 1st and 5th generation respectively (n=59). Groups of rats implanted with biopsies from four different patients (p1-p4). All bars=100 μm. Experimental methods are as in Example 11.

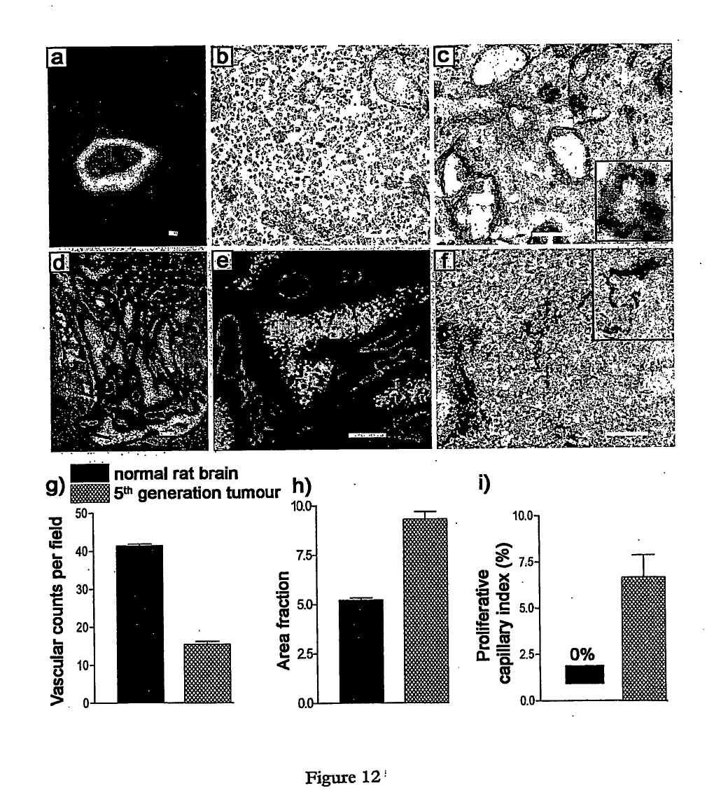

FIG. 12 shows 5th generation tumours show proliferating endothelial cells; (a) the PET uptake area displays a sharp border towards surrounding tissue indicating a more circumscribed lesion; (b) CD31-staining of the tumour bed; (c) co-staining of the tumour bed with CD31 (red) and Ki67 (brown), showing proliferating endothelial cells (inserted); (d) overview picture of the tumour with triple-staining against Pimonidazol (hypoxia), Collagen IV (red) and Hoechst (blue); (e) at high magnification hypoxic regions are seen surrounded by irregular dilated vessels with extravasation of Hoechst; (f) perfusion with Indian ink reveal leakage into the parenchyma from tortuous vessels (inserted); and (g-i) quantification of vascular parameters and comparison with normal brain. All bars=100 μm. Example 11 describes experimental methods;

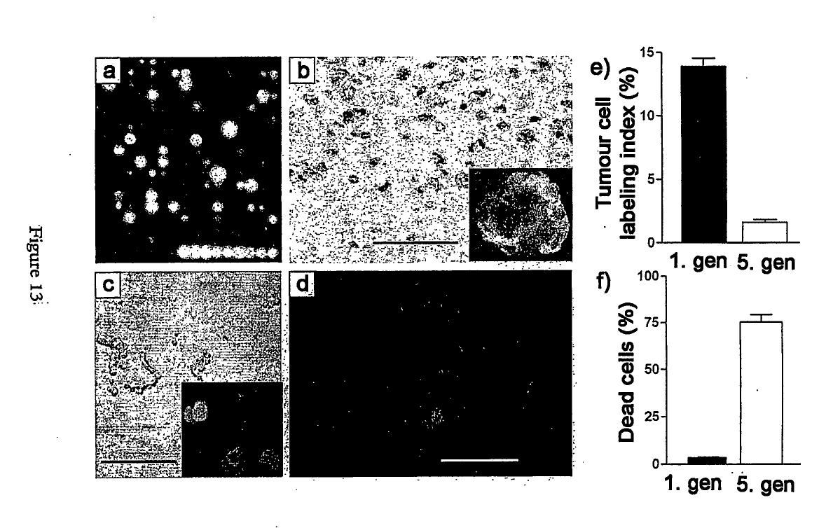

FIG. 13 shows loss of stem cell features in 5th generation tumours; (a) stem cell array with green and red spots representing genes upregulated in 1st and 5th generation, respectively. A majority of the spots are green or yellow indicating an upregulation of stem cell related genes in 1st generation tumours (yellow dots lower right represent housekeeping genes equally expressed in both generations); (b) immunostaining of 5th generation tumours show no nestin positive cells in the brain/tumour border zone. Also tumour explants stained in vitro for Musashi were negative (insert); (c) light microscopy of 5th generation tumour cells in stem cell medium showing deranged and fragmented cells without spheroid formation. Live-dead staining reveal numerous dead cells (red) with DAPI counterstain (insert); (d) Brdu-staining (red) of 5th generation tumour cells cultured in stem cell medium show few dividing cells, DAPI-counterstain (blue); and (e-f) comparison of the tumour phenotypes showing 1st generation tumours (1. gen.) to be viable and divide in stem cell medium whilst the majority of cells in the 5th generation tumours (5. gen.) die. All bars=100 μm;

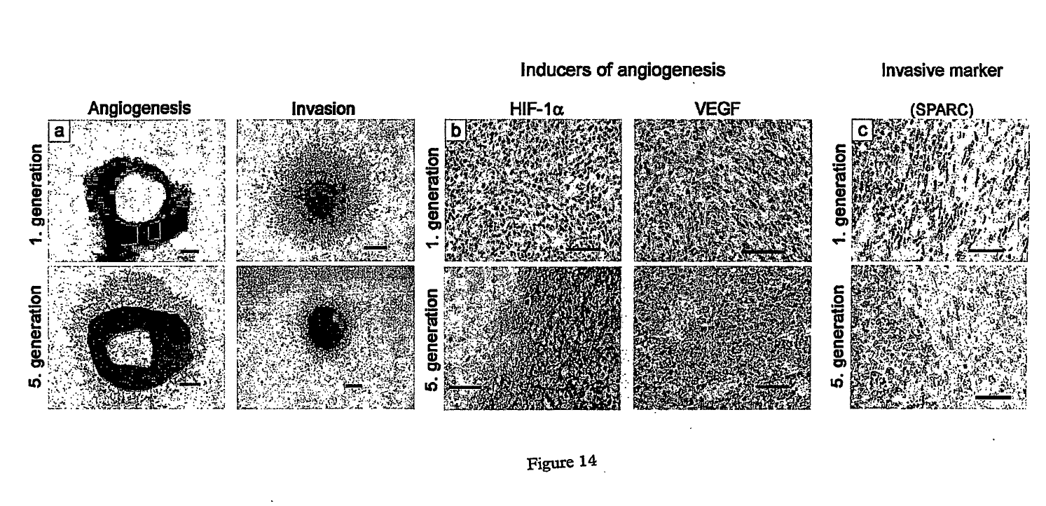

FIG. 14 shows angiogenesis and invasion assays showing an inverse relationship between angiogenesis and invasion; (a) No endothelial sprouting is seen from aortic ring explants when incubated with conditioned medium from 1st generation tumour spheroids (upper left). However, these spheroids display a strong invasion into the collagen gel assay (upper right). Medium incubated with tumour spheroids from 5th generation spheroids induces a strong endothelial sprouting (lower left), while the same spheroids display a limited invasive growth into the collagen gels (lower right). Pictures from aortic ring and collagen-invasion assays were all taken on day 3; (b) Hif-1α and VEGF expression is absent in 1st generation tumours and strongly expressed in the 5th generation tumours. The left picture in the lower panel shows Hif-1α expression in the tumour which disappear at the transition towards the surrounding brain; (c) SPARC is expressed in the invasive 1st generation tumours (upper), while the less invasive 5th generation tumours only displays a weak staining. All bars=100 μm. Example 11 describes the experimental protocols; and

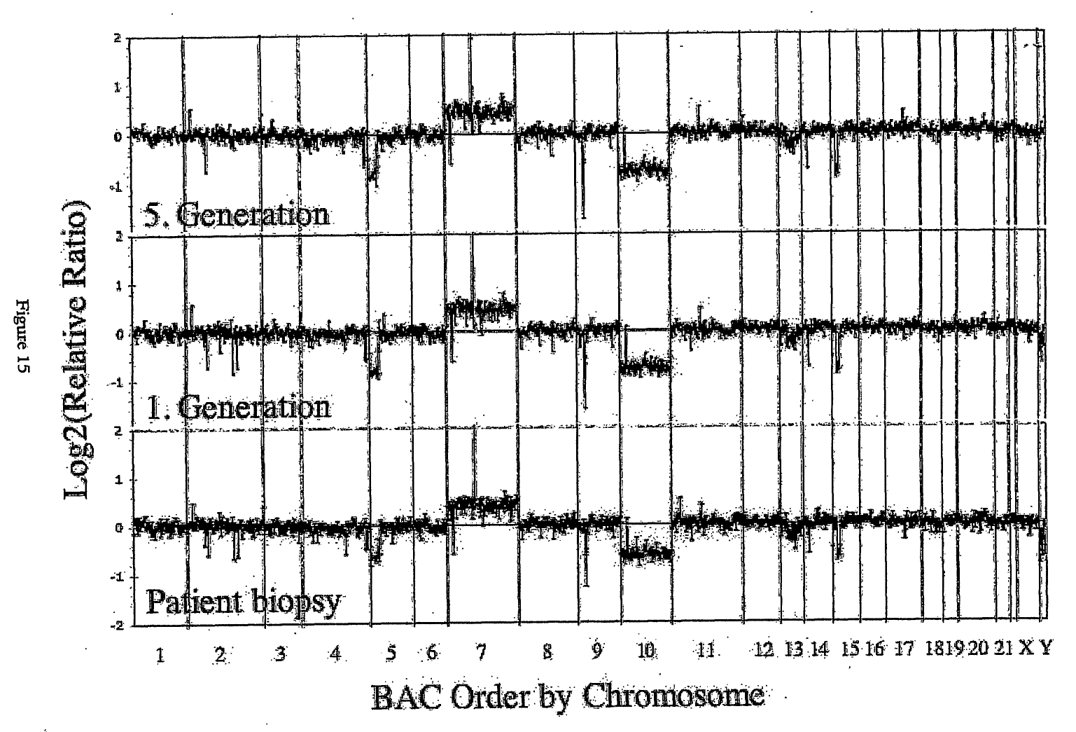

FIG. 15 shows the stem cell tumours and the angiogenesis-dependent tumours derived from them show genetic similarities to the parent tumour. Array CGH show a striking similarity in the relative chromosome copy numbers between the tumour phenotypes, indicating a close relationship between the human tumours and the tumours established in the rats. The results are plotted as mean log₂ ratio against BAC order by chromosome. The experimental method is set out in Example 11.

### EXAMPLES

### Example 1

### Collection of Tumour Tissue and Normal Brain Tissue from Patients, Culturing to Form Multicellular Spheroids

Fragments of tumour tissue (approximately 0.1 cm³) were obtained at surgery from sixteen patients with brain tumours. All the patients gave their verbal consent of tumour specimen collection for research purposes. The specimens were taken from regions with contrast enhancement on pre-operative computerized tomography scans and was macroscopically non-necrotic. This particular collection and use of tumour and normal brain tissue has been approved by the ethic board at Haukeland Hospital. All the tissue specimens were collected at Haukeland Hospital.

The tissue specimens were immediately transferred aseptically to a test tube containing Dulbecco's modification of Eagle's minimum essential medium (Gibco, Paisley, Scotland) supplemented with 10% heat-inactivated newborn calf serum, four times the prescribed concentration of non-essential amino acids and 2% L-glutamine, penicillin (100 IU/ml), and streptomycin (100 mg/ml) (DMEM). The tissues were maintained in culture as described below.

Tumour Tissue:

The tissue was cut with scalpels into pieces (approximately 0.1 mm³) and incubated in 80 cm² tissue culture flasks (Nunc, Roskilde, Denmark) in agar overlay culture. The culture flasks were base-coated with 10 ml of 0.75% agar (Difco, Detroit, Mich.) in DMEM. The volume of the overlay suspension was 12 ml and the DMEM was changed once every week. Culture took place in 80-sq cm tissue culture flasks. (Such conditions are as described in Bjerkvig et al, J. Neurosurg, Vol. 72, March 1990, 463 to 475). The DMEM overlay culture may be supplemented as required, e.g. with heat-inactivated fetal calf serum, non-essential amino acids, L-glutamine, penicillin and streptomycin. The spheroids were cultured in a standard tissue culture incubator (100% relative humidity, 95% air and 5% CO₂). Spheroids were cultured for 1 week (from excision) before transplantation into the nude rat brain in order to establish Phenotype I. To establish Phenotype III, the spheroids were maintained in culture for at least 6 weeks, before implantation. The size of the spheroids chosen for intracranial implantation was 100-300 μm.

### Example 2

### Intracranial Implantation of Multicellular Spheroids

Nude rats (Han:rnu/rnu Rowett) were bred in an isolation facility at 25° C. in a specific pathogen-free environment and humidified air (55% relative humidity) on a standard 12-hour night and day cycle. All animals were fed a standard sterilized pellet diet and provided sterile tap water ad libitum. All procedures and experiments involving animals in this study were approved by The National Animal Research Authority and conducted according to the European Convention for the Protection of Vertebrates Used for Scientific purposes.

All surgical procedures were performed on animals anaesthetised with pentobarbital at a concentration of 0.4 ml/100 g body weight, administrated intra peritoneally. Once anaesthetised, each rat was placed in a stereotaxic frame (David Kopf, model 900). A short incision was made in the skin, exposing the skull and allowing identification of the bregma point and the sagittal suture. The skull was trepanned using a high-speed microdrill with a bit diameter of 2.9 mm. The burrhole was located 1 mm caudal to the bregma and 3 mm lateral, and right to the sagittal suture. The dura mater was cross incised and 5 μl DMEM without serum containing 10 biopsy spheroids was injected using a Hamilton syringe with an inner diameter of 300 μm in which the piston reached the tip of the needle. The needle was kept at an angle of 90° to the skull during implantation and inserted 2.5 mm (from the dura mater) into the cortex of the brain and then slightly retracted, to allow room for the spheroids. The spheroids were injected over a period of two minutes and the needle was left in place for a further three minutes after injection. The needle was then slowly withdrawn from the brain and the skin was closed with 3.0 ethilon. After the inoculation, the animals were returned to their cages and observed until they recovered from the anaesthesia. The rats were observed daily and sacrificed by CO₂ inhalation when symptoms of intracranial disease appeared (symptoms of 1^{st} generation tumours). Symptoms consisted of passivity, clumsiness, and paresis. Brains were removed, washed in PBS, mounted on stubs, embedded in Tissue-Tec (Miles, Elkshart, N) and finally frozen in liquid nitrogen. Serial axial 10 μm sections were cut on a Reichert Jung Cryostat (Reichert, Vienna, Austria), and prepared for various histological screening assays as briefly described below. Some of the sections were stained with haematoxylin and eosin for light microscopic examination.

### Example 3

### Serial Transplantation In Vivo

Tumour tissue collected from the animals having 1^{st} generation tumours (Phenotype I) were dissected out under aseptically conditions and new biopsy spheroids were initiated according to the technique described above (1 week total culture period in vitro before reimplantation). The spheroids were then transplanted into the brains of new immunodeficient animals and the procedures were repeated four times giving rise to 2^{nd}, 3^{rd}, 4^{th} and 5^{th} generation tumours (Phenotype II). By doing this, we were able to follow tumour progression in vivo (Transition from Phenotype I to II), where the 3^{rd}, 4^{th} generation represent transitional or mixed phenotypes between Phenotype I and II.

### Example 4

### Tumour Analyses

Tumours were morphologically studied using by standard magnetic resonance imaging techniques, histology and immunohistochemistry, positron emission tomography scans, and transmission electron microscopy. Furthermore the tumours were assessed for the secretion of angiogenic factors using real time PCR techniques and western blots. The tumours (1^{st}-5^{th} generation tumours as obtained in Example 3) were also assessed for the expression of stem cell markers.

### Example 5

### Isolation of Transformed Human Stem Cell from the Rat Brain

Phenotype I:

We dissected out the 1^{st} generation tumours and transferred the tissue into a serum free stem cell medium containing the epidermal and fibroblast growth factors. Surprisingly the only cell type that survived in this medium was the transformed neural cell phenotype, which formed structures in vitro which could be associated with neurospheres. However, by reimplanting these neurospheres back in animals, according to the procedures described above, new tumours were formed, indicating that this cell population represent a true transformed cell population.

All Phenotypes

We have been able to obtain pure transformed cell populations that exhibit stem cell markers. We have also implanted these cells into the rat brain and observed that they give rise to Phenotype I tumours. Since the tumours grown in the laboratory animals represent human tumour cells growing in the animal brain, we have used a pan anti human antibody to extract the human transformed stem cells out from the animal brain tissue. This has been achieved by using both flow-cytometric cell sorting techniques as well as magnetic bead separation techniques. This technique was applied to isolate all phenotypes from the excised tissue.

### Example 6

### Results: Evidence for the Detection of an In Vivo Non-Angiogenic Invasive Tumour Cell Population (Phenotype I)

The brains of the animals from Example 2 were harvested, and the tumours were serially passaged in vivo, for five generations of rats as described in Example 3 (FIG. 1, left panel). Opposed to tumours derived from cell lines which grow as highly localized lesions within the brain, brains from rats in the first generation surprisingly displayed highly infiltrative tumours, both upon macroscopical inspection and histological Hematoxylin and Eosin (H/E) staining. Also to the inventors' surprise, no tumour vasculature and no formation of new vessels or areas of necrosis were seen; and thus the tumours were Phenotype I (FIG. 1 upper panels (also marked (b)). All subsequent generations were examined, and revealed that this invasive phenotype was essentially maintained. To the inventors' surprise they also observed that the propagation of these tumours according to Example 3 was accompanied by a gradual onset of angiogenesis, resulting in tumours with a disordered vasculature, enlarged vessels and endothelial cell proliferations; resulting in tumours of Phenotype II (FIG. 1, lower panels (also marked c)). Also, necrotic regions were clearly visible in the tumour areas. MRI-scans confirmed these findings as illustrated by an apparent change from diffuse non-enhancing lesions in 1^{st} generation (phenotype I, upper panels), to strongly contrast-enhancing tumours in the later generations; Phenotype II (FIG. 1 lower panels).

Brains from all generations were sectioned, and a panel of immunohistochemical stainings conducted to allow a more detailed analysis of the tumour vasculature. Normal brains were compared with tumours from 1^{st} and 5^{th} generation using the endothelial cell markers CD31 and von Willebrand (FIG. 2a). In tumours from the 1^{st} generation, the vessels had a structural morphology identical to the vasculature in normal brain tissue, with no significant difference in microvessel density (MYD) or vascular area. In the 5^{th} generation, vessels were irregular and markedly dilated with numerous endothelial cell proliferations. In this group, MVD was reduced as compared to normal brain and tumours of 1^{st} generation, while the total vascular area (TVA) was significantly increased.

Furthermore, Indian ink was injected to obtain a functional characterization of the vasculature in all groups (FIG. 2b). In tumours from the 1^{st} generation, functional capillaries appeared as those of normal brain with regular, small diameter vessels in the tumour parenchyma. In later generations, numerous large and irregular areas with Indian ink were detected, indicating a perfused, but chaotic vascular network.

We also performed triple stainings using the hypoxia marker Pimonidazol, Collagen IV and Hoechst in order to relate hypoxia to vascular morphology and function in the tumours (FIG. 2c). In the 1^{st} generation, we could not detect any hypoxia, and incorporation of Hoechst took place almost exclusively in close relation to the basal membrane marker Coll IV, suggesting an intact blood-brain barrier (FIG. 2c, left). In the 5^{th} generation, several hypoxic regions were visible in the tumour, and were surrounded by numerous enlarged and irregular vessels. In addition, Hoechst staining was evident outside the vessel lumens, in cellular clusters discrete from the basal membrane, indicating a disrupted blood-brain barrier with extensive leakage (FIG. 2c, middle and right).

Transmission Electron microscopy (TEM) supported these findings, showing a well defined basal lamina associated with the vasculature of 1^{st} generation tumours (2d). Also, tight junctions between endothelial cells were evident. In later generations, vessels appeared irregular with a poorly defined basal membrane.

Actively dividing cells were visualized in vivo, by injecting the animals with radio-labelled thymidine followed by Positron Emission Tomography (PET-scan) (FIG. 3a). The scans showed a diffuse uptake of radio-labelled thymidine, indicating a disseminated spread of tumour cells throughout the brain. Co-staining with Coil IV and BrdU displayed a similar picture, with dividing cells spreading locally from the injection site, as well as invading along the corpus callosum to the contra lateral hemisphere (FIG. 3b). Fluorescence activated cell sorting (FACS) confirmed the presence of mitotic cells in the primary biopsy as well as from the tumours at different generations (FIG. 3d). While the majority of cells in all specimens had a diploid DNA content, there was a relatively constant fraction of S-phase cells during passaging.

In order to study tumour growth over time, we performed repeated MR-scans at three different time points. These scans displayed diffusely growing lesions, accompanied by a progressive oedema which occupied most of the hemisphere in the terminal stage, causing a shift of midline structures (FIG. 3c).

Several lines of research now indicate that tumour angiogenesis is triggered by a hypoxia induced upregulation of VEGF. Since hypoxia was also detected in the angiogenic rat brain tumours, we wanted to see if this correlated with a similar upregulation of VEGF. In situ hybridization with mRNA on macro-arrays showed an upregulation of VEGF after in vivo passaging of the tumours (FIG. 4a). Quantitative-real time-per supported these findings as an 8 fold increase in VEGF was detected in the tumours of 5th generation as compared to 1st generation tumours (FIG. 4b).

Furthermore, while immunostaining for HIF-1α and VEGF was negative in tumour sections from the 1st generation, fifth generation tumours stained strongly positive for both markers (FIG. 4c). In order to confirm these findings, we also assessed these tumours angiogenic potential in an assay using rat aorta explants embedded in matrigel (FIG. 4d). After adding media from tumour spheroids of later generations to the aorta explants, endothelial cell sprouting was evident after 3 days in culture. In contrast, media from 1st generation tumour spheroids induced no outgrowth of endothelial cells for the whole observation period of 14 days.

### Example 7

In order to achieve Phenotype III tumours, the initial biopsy spheroids were maintained in culture for 6 weeks before transplantation to the rat brain. Surprisingly this gave rise to an angiogenic non-invasive phenotype. (FIG. 5). We also observed that these tumours as the Phenotype II tumours expressed significantly less neural stem cell markers as compared to the Phenotype I tumours.

### Example 8

### Evidence that the Tumour Phenotype I Cell Population Identified Actually Represent a Transformed Stem Cell Phenotype

Stem cells can be isolated from a variety of organs, as for instance from the skin and brain and they can be propagated in custom-made serum free medium supplemented with only fibroblast growth factor (FGF2) and epidermal growth factor (EGF). This medium will support the growth of stem cells but not the growth of differentiated cells as well as heterogenous tumour cells. Thus, growth media exist that is rather unique for the propagation of stem cells. By using such a “minimal” medium people have been able to isolate cells from malignant brain tumours that exhibit stem cell features, i.e. they are smaller than the other brain tumour cells and they will grow in the stem cell medium. However at present it is not clear if these cells actually represent true transformed stem cells or if the can give rise to tumours.

We observed that the 1^{st} generation (Phenotype I tumours) tumours exhibited an abundant expression of several neural stem cell markers as Nestin (FIG. 6). CD-133, Tuj-1, and 1′3-isoLM 1, which shows that this tumour actually consist of transformed stem cell phenotype. Thus, the growth of the tumour biopsy had selected out a tumour cell population that exhibit stem cell features. To further verify that the Phenotype I tumours consist of transformed neural stem cells, we dissected out the Phenotype I tumours and grew the cells in the serum-free stem cell medium as described above. We were by this approach able to show that the cell growth reflected the growth previously observed for normal stem cells grown as neurospheres (FIG. 7).

We were also able to show that the cells continued to express neural stem cell marker nestin, indicating that we were able to isolate out a true transformed stem cell population. By an enzymatic dissociation of the brains harbouring Phenotype I tumours into a single cell suspension, we were also able to sort out the transformed cell population using a fluorescence activated cell sorter or magnetic beads, using nestin as a selection marker. In addition, by reimplanting these cells into the rat brain, we were able to generate Phenotype I tumours.

### Example 9

### The Stem Cell Marker Nestin is Lost in the Transition from Phenotype I to Phenotype II and III Tumours

Immunostaining of Phenotype I, II and III tumours revealed a strong decrease in nestin staining from type I to type II and III tumours. This indicates that the Phenotype I tumours, which show a homogenous expression of stem cell markers can give rise to other tumour cell populations that do not express stem cell markers. The technique described, and which is schematically outlined in FIG. 8, represent therefore a unique tool for isolating different tumour cell population with different behavioural characteristics from a single brain tumour biopsy. The fact that the technique is highly controllable, makes it unique as a tool for in vivo gene discovery.

### Example 10

### Isolation of Cells from Pancreatic Tumours

Fresh pancreatic tumour tissue, obtained at surgery is immediately (within 20 minutes of excision) cut with scalpels into pieces (approximately 0.1 mm³) and incubated in 80 cm² tissue culture flasks (Nunc, Roskilde, Denmark) using an agar overlay culture method as earlier described. Briefly, the flasks were base-coated with 10 ml of 0.75% agar (Difco, Detroit, Mich.) in DMEM. The volume of the overlay suspension was 12 ml and the DMEM was changed once every week. The spheroids were cultured in a standard tissue culture incubator (100% relative humidity, 95% air and 5% CO₂.

The multicellular spheroids thus obtained may be transplanted into the pancreas, brain or liver of an immunodeficient animal. For transplantation into the brain the same procedure as used for the brain tumour biopsy spheroids may be used. Alternatively, for transplantation into the liver anaesthetised rats have a midline section made in the rat abdomen exposing the abdominal cavity. The pancreatic spheroids may then be injected into the liver using the same syringe as used for the brain. The size of the spheroids chosen for implantation is 100-300 μm.

### Example 11

This Example demonstrates that brain tumours have the capacity for angiogenesis-independent growth, mediated by a sub-population of transformed stem cells (cancer stem cells). These cells show an extensive invasion and cell division between existing vasculature. Tumours derived from the stem cell tumours will develop angiogenesis-dependency. The transition from angiogenesis-independent growth to angiogenesis-dependency is characterised by a down-regulation of pro-invasive genes and a loss of stem cell markers.

Experimental Procedures:

Cell culture: Biopsy spheroids were prepared as previously described (Bjerkvig et al., 1990, J Neurosurg 72, 463-475). After 1-2 weeks in culture, spheroids with diameters between 200 and 300 μm were selected for intracerebral implantation (see below). 1st and 5th generation tumour spheroids were cultured in parallel in a serum-free neural stem cell medium supplemented with EGF (20 ng/ml) and bFGF (20 ng/ml).

In vivo experiments: Nude immunodeficient rats (Han:rnu/rnu Rowett) were fed a standard pellet diet and provided water ad libitum. All procedures were approved by The National Animal Research Authority. Biopsy spheroids were stereotactically implanted into the right brain hemisphere as described elsewhere (Engebraaten et al., 1999, supra). The animals were sacrificed when symptoms developed and the brains were then removed.

MRI-imaging: MRI-image analysis was performed on a Siemens Magnetom Vision Plus1.5T scanner (Erlangen, Germany) using a small loop finger coil. Rats were anaesthetized and immobilized in a polystyrene tube. Coronal T₁ and T₂ images were obtained both before and after injection of contrast agent. A total of 19 coronal slices were obtained covering the brain. For details see Thorsen et al. (2003, J. Neurooncology, 63, 225-231).

PET-scans: The synthesis of [¹⁸F]FLT was performed as previously described (Shields et al., 1998, Nature Medicine, 4, 1334-1336), at the Radionuclide Centre (RNC) Amsterdam. 1 ml [¹⁸F]FLT was injected in the carotid arteries of four animals, and emission scans were performed at 45 minutes post injection of 18.5 MBq using a prototype single crystal high research resolution tomograph (HRRT) 3D PET scanner (CTI, Knoxville, Tenn.), with a resolution of 2.6 mm. Emission data were collected for 30 minutes and reconstructed using FBP.

Immunohistochemistry: Antibodies used were: anti-BrdU (Caltag Laboratories, Burlingame Calif.), anti-rat CD31 (diluted 1:1000, Pharmingen, San Diego, Calif.), Collagen IV (diluted 1:500, Dako, Glostrup, Denmark), anti-human HIF-1α (diluted 1:100, BD Biosciences, San Diego, Calif.), anti-human Ki67 (Dako), anti-rat Ki67 (Dako, diluted 1:100), anti-pimonidazol (Jackson Laboratories, West Grove Pa.), anti-human Musashi-1 (diluted 1:200, Chemicon, Temecula, Calif.), anti-human VEGF (Santa Cruz Biotechnology, Santa Cruz, Calif.), anti-human Vimentin (diluted 1:500, Dako) and anti-human von Willebrand factor (diluted 1:500, Dako). BrdU labelling was performed as previously described (Taki et al., 1994, J. Neurooncology, 19, 251-258), but no HCl treatment was performed. Nuclei were stained with Vectashield containing DAPI (Vector Labs, Burlingame, Calif.). Peroxidase and Alkaline Phosphatase reactions were performed on the sections using the En Vision+ Systems from DAKO, with the exception of CD31 which was stained using the animal research kit (ARK) from DAKO. Hoechst; BrdU and Pimonidazol were given systemically through the tail veins of the animals prior to sacrifice.

Live/dead staining: Cells were stained in Live/Dead Red. BioHazard Viability Kit (Molecular Probes, Eugene, Oreg.) for 20 minutes and fixed in PBS with 4% glutaraldehyde. Nuclei were stained with Vectashield containing DAPI.

Assessment of angioarchitecture: Normal brains as well as tumours from 1st and 5th generation stained for CD31 were inspected for areas with high micro vessel density (MVD) at ×4 magnification. In each brain, 25 regions (5 visual field in 5 areas) were selected for a closer analysis at ×400 magnification. Three independent observers performed this procedure providing 75 fields in each group for analysis. For image acquisition, the observers set a threshold to distinguish vascular elements from surrounding tissue, which were then assessed using LUCIA morphometry software from Nikon.

Transmission electron microscopy: The rats were perfusion fixed using 2% glutaraldehyde in 0.1M cacodylate buffer with 0.2M sucrose for at least 1 h (pH 7.2; 300±10 mOsmol). The brains were then removed and placed in the same fixative for 2 days. Tumours pieces were post-fixed for 1 hour in 1% OsO₄ and dehydrated in increasing concentrations of ethanol to 100%. Embedding in Epon 812 (Fluca, Buchs, Switzerland) was performed using graded additions of Epon-propyleneoxide mixtures. The polymerization was carried out at 60° C. for 24 hours. Ultratin sections were cut on a Leica Ultracut Microtome (Leica Microsystems, Bensheim, Germany) double-stained with uranyl acetate and lead citrate and examined by a Philips EM 410 transmission electron microscope (Philips, Eindhoven, the Netherlands).

Western-blot: CSF was run on SDS-PAGE. The primary antibody (rabbit anti-pan VEGF-A 1:100, Abcam, Cambridge, UK) was detected using a horse radish peroxidase (HRP) conjugated secondary antibody (goat anti-rabbit 1:20000) (Immunotech, Fullerton, Calif.).

Real time-rt-PCR: 0.5 μg total RNA was reverse transcribed using oligo dT-primers (Reverse Transcription Core Kit, Eurogentec, Philadelphia, Pa.), before running the PCR reaction in 25 μl volume with 1. 25 mM MgCl₂, 100 μM dNTP, 250 nM primer, 2 μl cDNA:RNA hybrid mixture and 0.625 units polymerase (Eurogentec) using the Smart Cycler System (Cepheid, Sunnyvale, Calif.). Forward and reverse primers for VEGF (26) and GAPDH primers (Eurogentec) were used. PCR products were analyzed electrophoretically in 1% agarose gels.

Aorta-ring assay: Thoracic aortas were removed from sacrificed rats, transferred to a petri dish with cold PBS with 2.2% glucose before fibro-adipose tissue was removed with micro-dissecting forceps. Aortas were cut into 1 mm segments embedded in growth factor reduced matrigel matrix (BD, Bedford, Mass.) and transferred individually to matrigel coated 24 well plates (Nunc AS, Roskilde, Denmark). Conditioned media were harvested from 1st and 5th generation tumour spheroids and added to the aorta explants twice daily. Endothelial sprouting was assessed daily by light microscopy during the observation period of eleven days.

Collagen-invasion-gel assay: The collagen solution was prepared by mixing 3.2 mg/mL collagen type 1 in 0.012M HCl and 10-fold concentrated DMEM (without FBS or antibiotics, the pH was adjusted using 0.1M NaOH). 500 μL of this solution was added to 24-well plates. Spheroids were embedded in the collagen matrix before gelation at 37° C. and 5% CO₂, the gel was overlaid with 500 mL supplemented DMEM.

Gene expression analysis: The human angiogenesis and tumour metastasis arrays from the Gearray Q series and the Gearray S series human stem cell Gene array (Superarray, Bethesda, Md.) was used for analyzing gene expression in angiogenic and non-angiogenic tumours derived from two patients. Total RNA was extracted using the RNeasy midi kit from Qiagen (Qiagen GmbH, Hilden, Germany). After biotin labeling, the cDNA was hybridized to the gene array membranes, followed by post hybridization washes. Images were acquired using a Fujifilm LAS 1000 luminescent image analyzer (Fujifilm, Medical Systems USA) and processed with the software Scanalyse2 (Michael Eisen, Stanford University, Calif.).

Array CGH. To determine the copy number across all chromosomes, we did comparative genomic hybridizations on whole-genome arrays of approximately 2,400 chromosomally mapped BAC clones (Hum.Arrayl.14) following previously described methods (Snijders et al., 2001, Nature Genetics, 29, 263-264). Briefly, we hybridized arrays simultaneously with 600 ng each of tumour DNA labelled with Cy3-dCTP by random priming and Cy5-labelled reference DNA from normal brain tissue. We counterstained the spotted BAC DNA with 4′.6′-diamidino-2-phenylindole hydrochloride (DAPI) and collected and processed the images of the three fluorochromes using custom software (Jain et al., 2002, Genome Res, 12, 325-332) that calculates the raw ratios and the mean log₂ ratios of triplicates of tumour to reference DNA hybridization. After normalization, we plotted mean log₂ ratios and analyzed the resultant graphs for deletions and gains along each chromosome (FIG. 15).

Results

Vessel Cooption can Mediate Aggressive Disease without Angiogenesis

Tumour spheroids established directly from 10 human glioblastoma biopsies were implanted in the brains of nude rats (Engebraaten et al., 1999, J Neurosurgery 90, 125-132; Mahesparan et al., 2003, Acta Neuropathol (Berl) 105, 49-57). Five months after implantation the animals developed neurological symptoms. They were then infused with ¹⁸F-3′-deoxy-3′-fluorothymidine (¹⁸[F]FLT) and examined by Positron Emmision Tomography (PET) (Shields et al., 1998, Nat Med 4, 1334-1336). The scans showed diffuse intracranial uptake of ¹⁸[F]FLT radio-labelleled thymidine, indicating a disseminated spread of dividing tumour cells throughout the brain (1st generation tumours; FIG. 9a), also invading the contralateral hemisphere (FIG. 9a). The PET results were verified by brain sections from rats that had been pulsed with bromodeoxyuridine (BrdU) prior to sacrifice. Dividing BrdU-positive cells were seen spread through the corpus callosum to the contralateral hemisphere (FIG. 9b). Moreover, triple staining for the basement membrane marker collagen IV and BrdU in rats that also had received a systemic injection of Hoechst 33342, revealed dividing cells between blood vessels with no Hoechst leakage into the surrounding parenchyma. This indicates normal vasculature among dividing tumour cells (FIG. 9c). We validated these results by Ki67/CD31 immunohistochemistry showing dividing Ki67 positive tumour cells among quiescent (FIG. 9d) normal sized blood vessels (FIGS. 9e and 9f). Brain sections of rats perfused with Indian ink (FIGS. 9g and 9h) and transmission electron microscopy also revealed a normal endothelial morphology with tight junctions between the endothelial cells (FIG. 9i). The area fraction representing vascular elements and vascular counts per field was slightly lower in the tumours compared to the normal brain (FIGS. 9j and 9k). This is consistent with tumour cells infiltrating the vascular bed, thus increasing the distance between neighboring vessels. No dividing endothelial cells were observed in the tumours (FIG. 9l).

Angiogenesis-Independent Tumour Growth is Mediated by Tumour Cells that Display Stem-Cell Characteristics

We repeated magnetic resonance imaging (MRI) at three different time points to study tumour progression (FIG. 10a). The T2 scans displayed diffuse lesions that occupied most of the hemispheres in the terminal stage, causing a shift of midline structures. Brains harvested from other rats in the same groups at the time of MRI allowed comparison with histological sections from corresponding regions (FIG. 10b). We identified dividing tumour cells in all regions of the brain using a human-specific antibody against vimentin (VIIIa et al., 2000, Exp Neurol, 161, 67-84), co-stained with Ki67 (FIG. 10c). The tumour cells, which were seen migrating along the corpus callossum, also expressed the neural stem cell marker nestin (Dahlstrand et al., 1992, Cancer Research, 52, 5334-5341). For comparison, nestin positive human neural stem cells (HNSC 100) showed a striking similarity to the tumour transplants in their migratory pattern (FIG. 10e). The tumours also expressed the neural stem cell marker Musashi (FIG. 10g), an RNA-binding-protein involved in asymmetric cell division during Drosophila neural development (Okabe et al., 2001, Nature, 411, 94-98). To further investigate the tumour's stem cell character, we incubated a single cell suspension from tumours in an EGF and FGF supplemented serum free medium which only neural stem cell growth (Calhoun et al., 2003, Biochem Biophys Res Commun, 306, 191-197). After 2 days, numerous cell clusters were seen indicating clonal growth (FIG. 10h). The cell clusters grew into viable spheroids (FIG. 10i) and incorporated BrdU indicating active cell division (FIG. 10j). When such spheroids were transplanted into the brains of nude rats, tumours developed, excluding the involvement of neural stem cells or stromal cells.

Angiogenesis-Independent Stem Cell Tumours, are the Source of Angiogenesis Dependent Tumour Clones

The tumour biopsies from four patients were serially passaged in the rat brain for five generations (5th generation tumours; FIG. 11a). Brains from rats in the 1st generation displayed highly infiltrative tumours, both upon macroscopic inspection and histologic H/E-staining. They showed neither pathologic tumour vasculature nor areas with necrosis (FIGS. 11b and 11c). The hemispheres were diffusely enlarged with no defined host-tumour border. In subsequent generations (FIG. 11d), the tumours became more circumscribed as the invasive phenotype gradually decreased. This was accompanied by a gradual onset of angiogenesis, resulting in tumours with disordered vasculature, enlarged vessels and endothelial cell proliferation. In addition, necrotic regions were clearly visible within the tumours (FIGS. 11c and 11d). Moreover, MRI-scans showed a transition from diffuse non-enhancing tumours in the 1st generation, to strongly contrast-enhancing lesions in the 5th generation tumours (FIGS. 11b and 11d, lower panels). Analysis of the cerebrospinal fluid from the rats revealed the presence of the vascular endothelial growth factor (VEGF) only in 5th generation tumours (FIG. 11f). The phenotypic shift from non-angiogenic to angiogenic tumours coincided with a significant decrease in survival from 113±26 (SD) to 43±11 days (SD) (FIG. 11g). Compared to the 1st generation tumours, the thymidine PET scans revealed more circumscribed tumours in the 5th generation (FIG. 12a), and these tumours displayed irregular and markedly dilated vessels and endothelial cell proliferation (FIGS. 12b and 12c). Triple staining for collagen IV and Pimonidazole in rats infused with Hoechst 33342 revealed numerous large hypoxic areas and dilated vessels with Hoechst leaking into the surrounding parenchyma (FIGS. 12d and 12e). This leakage was also confirmed in rats that had received systemic injections of Indian ink (FIG. 12f). A morphometric assessment of the vascular parameters revealed a lower vascular count per visual field in the 5th generation tumours (FIG. 12g), whilst the area fraction representing endothelial cells per visual field was increased (FIG. 12h). Finally, the proliferative capillary index was 6% in the tumours compared to 0% in the normal brain (FIG. 12i). All bars=100 μm.

Angiogenesis-Dependent Growth after Serial Animal Passage is Characterized by a Reduction in Stem Cell Markers

Stem cell cDNA microarrays, displaying 266 known genes that encode markers expressed by stem cells at various stages of differentiation, revealed a major upregulation of stem cell related genes in 1st generation compared to 5th generation tumours, including vimentin and nestin (FIG. 13a). The 5th generation tumours did not express nestin and Musashi (FIG. 13b) and the cells died when cultured in stem cell medium (FIG. 13c). The tumour cell labelling index fell from 14 to 1.6% when cultured in stem cell medium (FIGS. 13d and 13e), and the percentage of dead cells rose from 3.3 to 75% (FIG. 13f). These results indicate that new clones established during in vivo asymmetric tumour stem cell growth, exhibit classical tumour growth pattern, i.e angiogenesis dependent growth.

Non-Angiogenic Tumour Stem Cell Growth is Characterised by an Upregulation of Proinvasive Genes

Several lines of evidence indicate that tumour angiogenesis is triggered by hypoxia induced upregulation of VEGF (Plate et al., 1992, Nature 359, 845-848; Pugh and Ratcliffe, 2003, Nature Medicine, 9, 677-684). Since hypoxia was only detected in the angiogenic tumours, we investigated whether it corresponded with VEGF upregulation. The cDNA micro-arrays showed upregulation of VEGF and other angiogenic factors in the 5th relative to 1st generation tumours (Table 1). In contrast, a battery of pro-invasive genes was upregulated in the 1st relative to 5th generation tumours (Table 1). Quantitative-real time-PCR revealed an 8 fold increase in VEGF-mRNA in the 5th as compared to 1st generation tumours. In order to functionally confirm the differences in gene expression profiles, we assessed the angiogenic potential of 1st and 5th generation tumours in a rat aortic ring assay (FIG. 14a, left panels). Endothelial cell sprouting was only evident from aortic rings that received conditioned medium from 5th generation tumour spheroids. Conditioned media from 1st generation tumour spheroids induced no outgrowth of endothelial cells during the observation period of 11 days, suggesting that 1st generation tumours do not secrete the necessary amounts of angiogenic factors to trigger angiogenesis. Conversely, spheroids from 1st generation tumours were highly invasive when tested in a Collagen-invasion-gel assay, while the 5th generation tumour spheroids only displayed a modest invasion in the collagen gel (FIG. 14a, right panels).

We verified the gene expression profiles at the protein level. Immunostaining for HIF-1α and VEGF were negative in sections from 1st generation rat brain tumours (FIG. 14b, upper right and left), whereas staining for both markers were positive in the 5th generation tumours (FIG. 14b, lower right and left). The invasion marker SPARC (Schultz et al., 2002, Cancer Research, 62, 6270-6277) was up-regulated in 1st generation tumours whereas the 5th generation tumours displayed weak staining (FIG. 14c).

The Stem Cell Tumours Show Genetic Similarities to Human Gliomas

Array comparative genomic hybridization showed that the human biopsy and the early and late stage transplants had nearly identical genetic profiles. The human tumour biopsies and the phenotypes established in the rats showed a loss on chromosome Sp, gain on 7 with EGFR amplification, INK4A/ARF homozygous deletion, loss of chromosome 10 and interstitial loss of 15q (FIG. 15). The results show that the tumours derived from the rats are a good genetic representation of tumour cell populations in humans. Furthermore, the striking similarities in the CGH profiles between the tumours indicate that transcriptional regulation is an important component of the phenotypic differences seen in the model.

Summary

The results presented herein show that human cancer stem cells drive tumourigenesis through a distinct non-angiogenic highly invasive phenotype. This phenotype mediated a fulminant disease course, and was established from every human glioma xenotransplanted to the rat brain. This indicates that our observations are universal, and that angiogenesis is not a prerequisite for tumour growth. The non-angiogenic phenotype has the capacity to self-renew and expresses stem cell markers. However it is not yet clear whether these cells are directly derived from neural stem cells.

The fact that it takes at least five months for the human brain tumours to establish themselves in the rat brain, indicates that only a small fraction of the transplanted cells are adaptable enough to initiate tumour growth. The uncoupling of invasion and angiogenesis, represented by the cancer stem cells and the cells derived from them respectively, points at two different mechanisms that drive tumour progression. The results showing that both mechanisms can mediate a fulminant disease course, indicates that an effective cancer treatment strategy will need to pursue both the invasive stem cell as well as angiogenic targets.

TABLE 1
Differently expressed genes between
1st and 5th generation tumours

| | | |
|---|---|---|
| Gene | Unigene | Function |

Upregulated in 1st generation:

| | | |
|---|---|---|
| Secreted protein and rich | Hs. 173594 | Promotes glioma invasion |
| in cystein (SPARC) | | in vivo |
| Laminin B1 chain | Hs. 82124 | Provides substrate for |
| (Laminin B1) | | migrating glioma cells |
| Laminin gamma 1 | Hs. 214982 | Provides substrate for |
| (Laminin B2) | | migrating glioma cells |
| Integrin alpha 5 | Hs. 295726 | Integrin subunit involved |
| (Integrin αᵥ) | | in cell migration and |
| | | angiogenesis |
| Fibronectin-1 | Hs. 287820 | Provides substrate for |
| | | migrating glioma cells |
| Nestin | X 65964 | Neural stem cell marker |
| Vimentin | Hs. 297753 | Neural stem cell marker |

Upregulated in 5th generation:

| | | |
|---|---|---|
| Vascular endothelial growth | Hs. 73793 | Promotes angiogenesis |
| factor (VEGF A) | | |
| Vascular endothelial growth | Hs. 79141 | Promotes angiogenesis |
| factor C (VEGF C) | | and lymphangiogenesis |
| Platelet derived growth | Hs. 37040 | Subunit in PDGF AB |
| factor alpha polypeptide | | which induces VEGF |
| (PDGFA) | | expression |
| Platelet derived growth | Hs. 74615 | Receptor subunit for |
| factor receptor alpha | | PDGF-AA, PDGF-AB |
| polypeptide (PDGFAr) | | and PDGF-BB |
| Fibroblast growth factor | Hs. 748 | Mediates maturation of |
| receptor 1 (FGFr-1) | | endothelial cells |

1. A method of generating cells of a defined tumour phenotype, being invasive and angiogenesis-independent (phenotype I), from a tumour sample, said method comprising the steps of culturing tumour cells of said tumour sample for up to nine days in order to establish multicellular spheroids, and implanting said multicellular spheroids thus obtained into an immunodeficient animal.

2. The method of claim 1, wherein said tumour cells are cultured for up to seven days.

3. A method of generating cells of a defined phenotype, being invasive and angiogenesis-dependent (phenotype II), from a tumour sample, said method comprising the steps of:
(i) culturing tumour cells from said tumour sample in order to establish multicellular spheroids;
(ii) implanting said multicellular spheroids into an immunodeficient animal;
(iii) allowing a tumour to develop, in the case of this first implantation step, said tumour being invasive and angiogenesis-independent (phenotype I);
(iv) isolating a tumour sample or tumour cells from said animal;
(v) repeating steps (i) to (iv) until the tumour becomes angiogenesis-dependent.

4. The method of claim 3 wherein steps (i) to (iv) are repeated 1 to 10 times.

5. The method of claim 3 wherein said tumour cells are cultured as multicellular spheroids for 1 day to 6 weeks.

6. The method of claim 3, wherein in the first tumour cell culture step of step (i) to establish a tumour of phenotype I, the tumour cells are cultured for up to 21 days.

7. The method of claim 3, wherein in the first tumour cell culture step of step (i) to establish a tumour of phenotype I, the tumour cells are cultured for up to 9 days.

8. A method of generating cells of a defined tumour phenotype, being non-invasive and angiogenesis-dependent (phenotype III) from a tumour sample, said method comprising the steps of culturing tumour cells of said tumour sample for 5 to 10 weeks in order to establish multicellular spheroids, and implanting said multicellular spheroids thus obtained into an immunodeficient animal.

9. A method for generating cells of phenotypes I, II and III from a tumour sample, said method comprising the steps of culturing tumour cells of said tumour sample in order to obtain multicellular spheroids, wherein (a) to obtain cells of phenotype I the tumour cells are cultured for up to 21 days, and wherein (b) to obtain cells of phenotype III, the cells are cultured for 5 to 10 weeks, and implanting said multicellular spheroids into an immunodeficient animal, and wherein (c) to obtain cells of phenotype II, the method comprises the steps of
(i) isolating cells of phenotype I from said animal;
(ii) culturing said cells in order to obtain multicellular spheroids;
(iii) implanting said multicellular spheroids into an immunodeficient laboratory animal;
(iv) allowing a tumour to develop in said animal;
(v) isolating tumour cells from said animal;
(vi) culturing the tumour cells in order to obtain multicellular spheroids, and
(vii) repeating steps (iii) to (vi) until the tumour implanted into said animal becomes angiogenesis-dependent.

10. The method of claim 1 wherein said tumour cells are allowed to develop in said animal into a tumour.

11. The method of claim 1 wherein said tumour sample is from a human.

12. The method of claim 1 wherein said culturing step is performed in overlay culture medium.

13. The method of claim 1 wherein said immunodeficient animal is a rodent.

14. The method of claim 1 wherein said spheroids are implanted into a highly vascularised organ in said animal.

15. The method of claim 1 wherein said spheroids are 100 to 300 μm in diameter.

16. The method of claim 1 wherein up to 20 spheroids are implanted in said animal.

17. The method of claim 1 wherein said tumour sample is a brain tumour sample.

18. The method of claim 17 wherein said spheroids are implanted into the brain of an immunodeficient animal.

19. The method of claim 1 wherein said tumour sample is a pancreatic tumour sample.

20. A method of isolating cells of a defined tumour phenotype, being invasive and angiogenesis independent (phenotype I), said method comprising generating tumour cells of said phenotype as defined in claim 1, and isolating tumour cells of said phenotype from said animal.

21. A method of isolating cells of a defined tumour phenotype, being invasive and angiogenesis dependent (phenotype II), said method comprising generating tumour cells of said phenotype as defined in claim 3, and isolating tumour cells of said phenotype from said animal.

22. A method of isolating cells of a defined tumour phenotype, being non-invasive and angiogenesis dependent (phenotype III), said method comprising generating tumour cells of said phenotype as defined in claim 8, and isolating tumour cells of said phenotype from said animal.

23. A method of isolating cells of phenotypes I, II and III from a tumour sample, said method comprising generating said cells by a method as defined in claim 9 and isolating tumour cells of said phenotypes from each said animal.

24. Cells of a defined tumour phenotype, being phenotype I obtainable by the method of claim 20.

25. A method for generating an animal model of a defined tumour phenotype, being invasive and angiogenesis independent (phenotype I) comprising the steps as defined in claim 1.

26. A method for generating an animal model of a defined tumour phenotype, being invasive and angiogenesis dependent (phenotype II), comprising the steps as defined in claim 3.

27. A method for generating an animal model with implanted tumour cells of a defined tumour phenotype, being non-invasive and angiogenesis dependent (phenotype III), comprising the steps as defined in claim 8.

28. A method for generating an animal model with a tumour of phenotype II, or an intermediate or mixed phenotype between phenotype I and phenotype II, from a tumour sample, said method comprising:
(i) culturing tumour cells from said tumour sample in order to obtain multicellular spheroids;
(ii) implanting said spheroids into an immunodeficient animal;
(iii) allowing a tumour to develop, in the case of this first implantation step, said tumour being invasive and angiogenesis-independent (phenotype I);
(iv) isolating a tumour sample or tumour cells from said animal;
(v) repeating steps (i) to (iv) one or more times wherein to obtain an animal model of phenotype II, said steps are repeated until the tumour becomes angiogenesis-dependant.

29. An animal model obtainable by the method of claim 25.

30. An animal model of a tumour of phenotype I (angiogenesis-independent and invasive), said animal comprising implanted tumour cells or a tumour derived therefrom, wherein at least 75% of said tumour or tumour cells is or are of phenotype I.

31. An animal model of a tumour of phenotype II (angiogenesis-dependent and invasive), said animal comprising implanted tumour cells or a tumour derived therefrom, wherein at least 75% of said tumour or tumour cells is or are of phenotype II.

32. An animal model of a tumour of phenotype III (angiogenesis-dependent and non-invasive), said animal comprising implanted tumour cells or a tumour derived therefrom, wherein at least 75% of said tumour or tumour cells is or are of phenotype III.

33. An animal model as claimed in claim 30 wherein said tumour or tumour cells are xeno-transplanted.

34. A method of studying tumour progression, said method comprising comparing at least two tumour cells of claim 24.

35. The method of claim 34 wherein the invasiveness, state of angiogenesis, and/or stem cell characteristics are compared.

36. A preparation of tumour cells of phenotype I, wherein said cells are invasive and angiogenesis independent, and wherein at least 75% of said cells are of phenotype I.

37. The preparation of cells of claim 36 wherein said cells are transformed stem cells and express at least one stem cell marker.

38. A preparation of tumour cells of phenotype II, wherein said cells are invasive and angiogenesis-dependent, and wherein at least 75% of said cells are of phenotype II.

39. A preparation of tumour cells of phenotype III, wherein said cells are non-invasive and angiogenesis-dependent, and wherein at least 75% of said cells are of phenotype III.

40. Use of the cells of claim 24 in determining differential gene and/or protein expression.

41. Use of the cells of claim 24 to identify therapeutic targets.

42. A method of generating or isolating a transformed tumour cell from a tumour sample, said method comprising generating or isolating a tumour cell of phenotype I as defined in claim 1.

43. A method of studying tumour progression, said method comprising comparing at least two tumours of the animal model of claim 29.
